(19) United States
(12) Patent Application Publication
(10) Pub. No.: US 2015/0234981 A1
(43) Pub. Date: Aug. 20, 2015

(54) System and Method for Determining Relatedness

(71) Applicant: eGenomics, Inc., New York, NY (US)

(72) Inventor: Steven Naidich, New York, NY (US)

(21) Appl. No.: 14/689,405

(22) Filed: Apr. 17, 2015

Related U.S. Application Data
(63) Continuation of application No. PCT/US2014/031056, filed on Mar. 18, 2014.
(60) Provisional application No. 61/794,042, filed on Mar. 15, 2013.

(51) Int. Cl. G06F 19/22; C40B 30/02; G06F 19/00; G06F 19/14

(52) U.S. Cl. CPC G06F 19/22; G06F 19/14; C40B 30/02; G06F 19/3493; C12Q 2600/16; C12Q 2600/156

(57) ABSTRACT

Methods of determining a source of, and/or tracking the transmission of, an organism, including pathogenic organisms. Processor-readable medium having processor-executable instructions for performing such methods. Systems for tracking the path of an infection. Electronic systems for tracking the transmission of a pathogen. Methods for determining regions of DNA suitable for one-way analysis. Infection Control Analysis Decision Systems comprising a processing device in communication with memory containing instructions for carrying out methods of determining a source of, and/or tracking the transmission of, an organism, including pathogenic organisms.

This application is a continuation of International Application No. PCT/US2014/031056 filed on Mar. 18, 2014 and claims the benefit of U.S. Application No. 61/794,042 filed on Mar. 15, 2013, both of which are incorporated herein in their entirety for all purposes. It is noted that Mar. 15, 2014 was a Saturday, Mar. 16, 2014, was a Sunday, and Mar. 17, 2014 was a day on which Federal Government offices in the Washington D.C. area, including the USPTO, were closed, and therefore a “Federal holiday within the District of Columbia.” See Closing of the United States Patent and Trademark Office on Monday, Mar. 17, 2014, 14 Off. Gaz. Pat. & Trademark Office 107 (May 13, 2014).

### BACKGROUND OF THE INVENTION

1. Field of the Invention

This application relates to systems and methods for determining relatedness, for example among organisms present in a healthcare facility.

2. Description of the Related Art

Clinical healthcare environments, such as hospitals and long term care facilities, occasionally perform epidemiological studies in order to identify clusters and recognize outbreaks of disease. Understanding pathogen distribution and relatedness is essential for determining the epidemiology of nosocomial infections and aiding in the design of rational pathogen control methods.

Whole genome DNA sequencing on a mass scale has become technically possible as well as affordable. However, whole genome sequencing results in so much genomic data that the resulting data analysis is unwieldy and impractical. Historically, epidemiological studies in healthcare facilities have incorporated molecular typing techniques to distinguish among isolates.

Techniques such as RFLP, ribotyping, PFGE, MLEE, bacterial barcodes, and even DNA sequencing techniques such as spa-typing or MLST are ineffective in most clinical scenarios because the techniques do not adequately distinguish among clonal, endemic pathogen strains commonly found in healthcare facilities. These aforementioned molecular typing techniques succeed in longer term epidemiological studies where samples are collected over a wider range of time and there is greater diversity among samples. However, in scenarios where isolates are collected within a short time frame, a new method to analyze very closely related organisms is required.

Traditional epidemiological studies retrospectively discover statistical clusters of disease. Clusters of disease may elucidate an originating source, which, once identified, may be eradicated to prevent future disease spread. Organizations such as the CDC and the World Health Organization have established guidelines for disease investigations that include data collection and statistical analysis protocols. Traditional epidemiological statistics require the collection of relevant amounts of data so that accurate conclusions can be drawn.

However, traditional epidemiological investigations lead to a posteriori conclusions because a statistically relevant number of patients must be infected before sufficient data is collected to make accurate conclusions. Such a posteriori analysis can help identify and correct problematic situations, such as controlling a disease outbreak that has already begun, but such a posteriori epidemiological studies are not able to prevent a disease outbreak from occurring in the first place.

There is a need in the art for a system and method for performing infection control that can effectively prevent pathogen spread before statistically relevant disease clusters appear. Such systems and methods are described herein.

### SUMMARY

Described herein are systems and methods to analyze very closely related entities, for example organisms, that can be described by a discrete state at a moment in time such as an organism. In these systems and methods the system and method is used to first track and then alter the spread of infectious organisms by determining whether a plurality of organisms are very closely related to each other, and then using this information to eradicate the source and/or alter the subsequent path of transmission.

In preferred embodiments, the system and method that 1) determines relatedness among very closely related organisms, and 2) applies such relatedness results into a healthcare clinical Infection Control Analytical Decision System that directs the actions of healthcare workers. The system and method of determining relatedness among closely related organisms can be accomplished using DNA sequencing and also all other phenotypic laboratory tests, such as those mentioned above, that express an organisms DNA in an output format other than the character based AGCT output from a DNA sequencer. This system and method will work with all laboratory tests whose output is an expression an organism's DNA.

In an embodiment, the invention is directed to a method of determining a source of, and/or tracking the transmission of, a pathogenic organism, the method comprising:
receiving, in a processing device, laboratory test results representing partial or complete nucleotide sequence or expression state data for a pathogenic organism in a first biological sample and in a second biological sample,
comparing, by a processing device, a genetic state data for the organism in the first biological sample to a genetic state data for the organism in the second biological sample;
determining, by the processing device, whether the first and second nucleotide sequence or expression states have a one-away relationship based on the partial or complete nucleotide sequence or expression state data for the pathogenic organism in the first biological sample and in the second biological sample;
recording, in memory in communication with the processing device, the relationship between the organism in the first and second biological samples if the first and second nucleotide sequences or expressions are the same or one-away; and
constructing, by the processing device, a representation of the transmission of the pathogenic organism based on connections between samples containing organisms having a one-away relationship.

In another embodiment, the invention is directed to a processor-readable medium having processor-executable instructions for performing a method comprising:
a) receiving a laboratory test result on DNA collected from a pathogenic organism in a first sample;
b) receiving a laboratory test result on DNA collected from from a pathogenic organism in a second sample;
c) if the result of the first laboratory test is identical to the result of the second laboratory test, then record that the two organisms are identical and stop;
d) if the result of the first laboratory test is not identical to the result of the second laboratory test, then analyze the two laboratory test results to determine whether the two laboratory test results are one-away by a method chosen from among
   i. comparing each laboratory test result to a database of previously analyzed laboratory test results, and if both laboratory test results are found in the database, then look up and output whether the test results are one event away or more than one event away and stop,
   ii. comparing each laboratory test result to a database of generated in silico test results, and if both laboratory results match in silico test results in the database, then look up and output whether the two in silico test results are “one event away” or “more than one event away” and stop, and
   iii. analyzing the laboratory test results to determine whether the two laboratory test results are one-away, then output the analysis result and stop.

In another embodiment, the invention is directed to a system for tracking the path of an infection comprising:
a memory for storing first and second nucleotide sequences or expressions of nucleotide sequences determined from a pathogenic organism present in a first and second biological sample;
a processor configured to:
   access the first and second nucleotide sequences or expression from the memory;
   compare the first and second nucleotide sequences or expressions;
   determine whether the first and second nucleotide sequences or expressions are the same, one-away, or not one-away;
   connect the first and second biological samples if the first and second nucleotide sequences or expressions are the same or one-away; and return a report of connected biological samples.

In another embodiment, the invention is directed to an electronic system for tracking the transmission of a pathogen, the system comprising:
a receiving device configured to receive a first laboratory test result on DNA collected from a pathogenic organism in a first sample and a second laboratory test result on DNA collected from a pathogenic organism in a second sample;
a processing device configured to
   compare a genetic state data for the organism in the first biological sample to a genetic state data for the organism in the second biological sample,
   store that the two organisms are identical if the result of the first laboratory test is identical to the result of the second laboratory test, or
   analyze the two laboratory test results to determine whether the first and the second laboratory test results are one-away if the result of the first laboratory test is not identical to the result of the second laboratory test,
   wherein the processor makes the determination whether the first and the second laboratory test results are one-away by one of
   comparing each laboratory test result to a database storing previously analyzed laboratory test results, and outputting whether the test results are one event away or more than one event away if both laboratory test results are found in the database,
   comparing each laboratory test result to a database of generated in silico test results, and outputting whether the two in silico test results are “one event away” or “more than one event away” if both laboratory results match in silico test results in the database, or
   analyzing the laboratory test results to determine whether the two laboratory test results are one-away, and outputting the analysis result.

In another embodiment, the invention is directed to a method for determining regions of DNA suitable for one-way analysis, the method comprising:
receiving, by a receiving device, a plurality of pathogens;
performing, by a processor, genome sequencing of the plurality of pathogens;
comparing, by the processor, genome sequence of each of the plurality of pathogens with the genome sequences of all of the other plurality of pathogens of a same species;
identifying, by the processor, a DNA sequence for a gene coding region, the gene coding region being present in each of the genome sequences of the same species;
storing, in a database, the DNA sequence for every gene present in every genome sequences of the same species;
identifying, by the processor, all gene coding regions substantially present in each of the genome sequences of the same species;
storing, in a database, the DNA sequence for every gene substantially present in every genome sequences of the same species;
identifying, by the processor, all regions of DNA of the same species having a variable number of tandem repeats;
storing, in a database, the DNA sequence for every region having the variable number of tandem repeats;
identifying, by the processor, all single nucleotide polymorphisms in a conserved region among the genome sequences for the same species;
storing, in a database, the DNA sequence for every identified single nucleotide polymorphisms and the surrounding conserved DNA;
comparing, by the processor, similar regions of DNA;
determining, by the processor, a number of identical sequences from comparable regions of DNA and a number of variations among the comparable regions of DNA; and
selecting, by the processor, a plurality of regions to identify “one-away” events based on the number of identical sequences from comparable regions of DNA and the number of variations among the comparable regions of DNA

In another embodiment, the invention is directed to an Infection Control Analysis Decision System comprising a processing device in communication with memory containing instructions for carrying out the method of claim 1 for a plurality of pathogens in a healthcare facility and instructions for applying Bayesian statistical techniques to calculate the likelihood that a patient will acquire an infection from a pathogen with a specific molecular fingerprint based upon patient risk factors and the spatial-temporal density of each pathogen and to output specific actions for preventing the transmission of the pathogens.

### BRIEF DESCRIPTION OF THE DRAWING FIGURES

FIG. 1 depicts a block diagram illustrating a system architecture suitable for implementing the system and methods described herein.

FIG. 2 illustrates an exemplary flow of information.

FIG. 3 illustrates applications of the systems and methods described herein.

FIG. 4 illustrates a computer system architecture for use in implementing the systems and methods described herein.

FIG. 5 illustrates data input schema.

FIG. 6 illustrates relationships between hypothetical closely related organisms.

FIG. 7 illustrates an exemplary process for determining regions of pathogen DNA that are suitable for one-away analysis.

FIG. 8 illustrates the collection and use of biological samples in the systems and methods described herein.

FIG. 9 illustrates a sequence one-away algorithm.

FIG. 10 illustrates an exemplary application of the system and methods described herein.

FIG. 11 illustrates an exemplary algorithm for generating a PFGE test result in silico.

FIG. 12 illustrates an algorithm for generating a database of DNA microarray in silico test results.

FIG. 13 illustrates an algorithm for generating a database of in silico generated possible sequences that are one-genetic event away from each other (a one-away database).

FIG. 14 illustrates a method for performing in silico PFGE tests using all known restriction enzymes.

### DETAILED DESCRIPTION OF THE INVENTION

Described herein are systems and methods to analyze very closely related entities that can be described by a discrete state at a moment in time, for example, an organism. In preferred embodiments, these systems and methods are used to first track and then alter the spread of infectious organisms by determining whether a plurality of organisms are very closely related to each other, and then using this information to alter the subsequent path of transmission. In preferred embodiments, the spread of an undesirable organism, such as a pathogen, can be traced to identify the source of the organism and mitigate the spread of the organism, for example, by identifying and quarantining or sterilizing sources of pathogen, or by identifying and quarantining or sterilizing or removing a transmission vector.

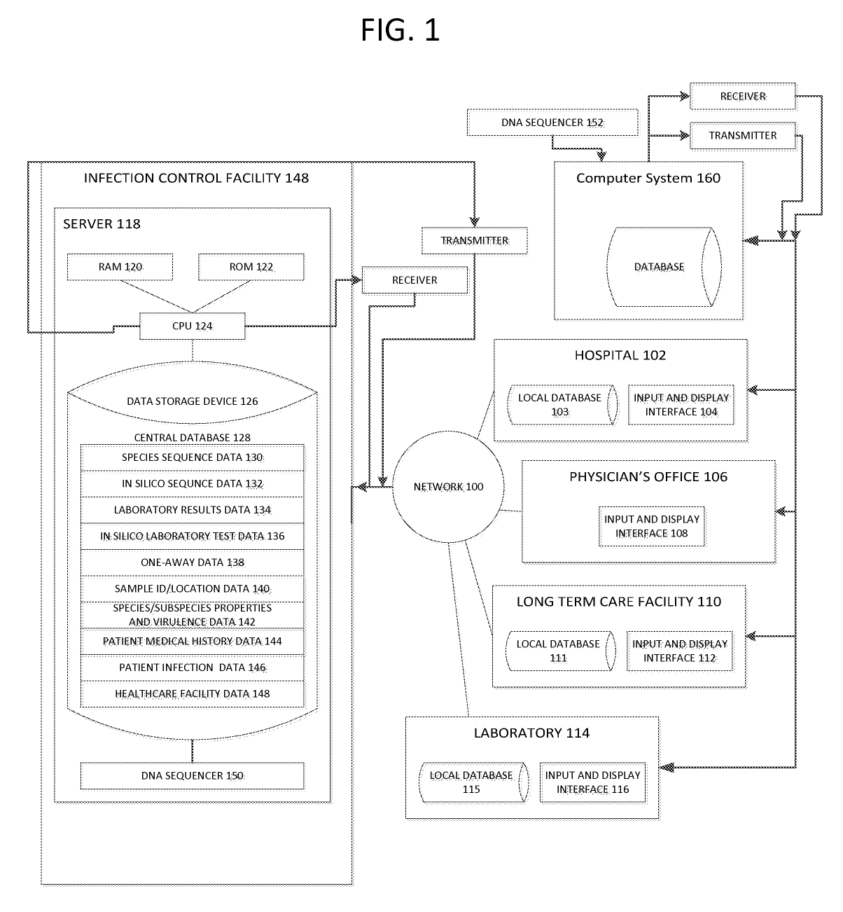

FIG. 1 depicts a blocking diagram illustrating a system architecture suitable for implementing the methods described herein. As shown in FIG. 1, various terminals at healthcare facilities such as hospital terminal 102, a physician's office terminal 106, long term care facility terminal 110, and laboratory terminal 114 can communicate with an infection control facility 148 via a network 100. Other institutions or entities involved in infection control can also connect to the facility 148 via network 100, for example a farm facility or other agriculture related environment, a food preparation facility, and an athletic facility such as a gym or training facility, etc..

Network 100 can be any network connecting computers. Network 100 can be a wide area network (WAN) connecting computers such as the Internet. Network 100 could also be a local area network (LAN). Hospital terminal 102, physician's office terminal 106, long term care facility terminal 110, and laboratory terminal 114 provide input and display interfaces 104, 108, 112 and 116, respectively. Some or all of these facilities may have a DNA sequencer 152, and other laboratory test equipment (not shown) which are connected to computer system(s) 160. A central DNA sequencer 150 and other laboratory test equipment can also be interfaced directly with the system 148.

Sequencers 150, 152 sequence predetermined regions of DNA from infectious isolates received from various healthcare facilities. Infection control facility 148 stores and analyzes the sequence data, tracks the spread of infections, and predicts infection outbreaks. Infection control facility 148 then informs the healthcare facilities of potential outbreak problems and provides infection control information.

Infection control facility 148 communicates with the local facilities via network 100. As an alternative to the use of a network, infection control facility 148 could communicate with the local facilities via alternative means such as fax, direct communication links, wireless links, satellite links, or overnight mail. Infection control facility 148 could also physically reside in the same building or location as the healthcare facility. For example, infection control facility 148 could be located within hospital 102. It is also possible that each of the remote healthcare facilities has its own infection control facility.

Infection control facility 148 includes a server 118. The server 118 contains a central processing unit (CPU) 124, a random access memory (RAM) 120, and a read only memory (ROM) 122. CPU 124 runs a software program for performing the methods described further below.

CPU 124 also connects to data storage device 126. Data storage device 126 can be any electronic, magnetic, optical, or other digital storage media. As will be understood by those skilled in the art, server 118 can be comprised of a combination of multiple servers working in conjunction. Similarly, data storage device 126 can be comprised of multiple data storage devices connected in parallel.

Central database 128 is located in data storage device 126. Central database 128 stores digital sequence data received from sequencers 150 and 152. Central database 128 also stores various types of information received from the various healthcare facilities. CPU 124 analyzes the infection data stored in central database 128 for infection outbreak prediction and tracking. Some examples of the various types of data that are stored in central database 128 are shown in FIG. 1. These types of data are not exclusive, but are shown by way of example only.

Species sequence data 130 stores the digital sequence data of an infectious agent such as a bacterium, virus, or fungus. This data can be used to determine specific regions to be investigated as described below. Different organisms will have different predetermined regions of their respective DNA that are sequenced for analysis. For example, an isolate of S. aureus bacteria will have different regions that are sequenced than an isolate of E. facaelis. Each type of bacteria or other infectious agent will have predetermined regions that are used for sequencing. The way that those predetermined regions are chosen is described in more detail below.

Sequences observed in various biological specimens are stored in observed sequence data 130. When an infectious isolate is obtained from a patient, other individual, or a piece of equipment, the DNA is sequenced in whole or in part and stored in DNA sequence data 130. Central database 128 can store any number of sequenced regions of the DNA. Data storage device 128 may also contain a database of in silico sequence data 132 generated as described below. The sequence data 130 may be compared to in silico sequence data 132 which represents pairs of sequences that are known to be one-away.

Laboratory test results that represent expressions of sequence data are stored in laboratory results data 134. These results may comprise, for example electrophoresis banding patterns and microarray data generated as described below. In silico laboratory test data 136 may be generated as described below and stored in central database 128.

Central database 128 also stores data records of previously observed one-away data 138, for example records of samples that have been previously identified as having a one-away relationship. The one-away data may be queried to determine if sequence or laboratory results data under consideration has previously been determined to have a one-away relationship.

Central database 128 also stores sample ID/location data 140 comprising time and place information for each sequence or laboratory result. It is desirable for the data storage device 128 to store the locations of patients, objects, healthcare workers and civilians even if those entities do not have an infection or sign of disease. Furthermore, the locations of these entities will be tracked and stored at multiple and regular time intervals. This will allow the system to calculate whether an uninfected patient is more likely to obtain an infection from a specific pathogen because the uninfected patient was moved to a location in closer proximity to another known pathogen source such as an infected patient, a contaminated object (known or unknown) or a colonized person. That other known pathogen source's location may also have moved from its original location and both the source and uninfected patient's path happened to become close in space and time during a period of time. This time and place data can be queried by CPU 124 for determination of whether two samples that are genetically one-away are related in time and place to a sufficient degree to be considered possibly related in a chain of transmission, particularly when constructing a network graph or phylogenetic tree for tracking the transmission and/or source of an infection.

Central database 128 also stores species/sub-species properties and virulence data 142. Data 142 includes various properties of different species and subspecies of infectious agents. For example, data 142 can include phenotypic and biomedical properties, effects on patients, resistance to certain drugs, and other information about each individual subspecies of microorganism.

Patient medical history data 144 contains data about patients such as where they previously have been hospitalized and the types of procedures that have been done. This type of data is useful in determining where a patient may have previously picked up an infectious agent, and determining how an infection may have been transmitted.

Patient infection information data 146 stores updated medical information pertaining to a patient who has obtained an infection. For example, data 146 could store that a particular patient acquired an infection in a hospital during heart surgery. Data 146 includes the time and the location that an infection was acquired. Data 146 also stores updated data pertaining to a patient's medical condition after obtaining the infection, for example, whether the patient died after three weeks, or recovered after one week, etc. This information is useful in looking for correlates between a disease syndrome and a strain subtype. Additional phenotypic assays to determine toxin production, heavy metal resistances and capsule subtypes, as examples, will also be added to the strain database and update properties and virulence data 142.

Healthcare facility data 148 contains information about various facilities communicating with server 118 such as hospital 102, physician's office 106, and long term care facility 110. Healthcare facility data 148 contains such information as addresses, number of patients, areas of infection control, contact information and similar types of information. Healthcare facility data 148 can also include internal maps of various healthcare facilities. As will be described later, these maps can be used to analyze the path of the spread of an infection within a facility.

Some of the healthcare facilities also have local databases. FIG. 1 shows that hospital 102, long term care facility 110 and laboratory 114 include local databases 103, 111, and 115, respectively. The local databases can store local copies of selected infection control information and data contained in central database 128, so that the healthcare facility can access its local database for infection control information instead of having to access central database 128 via network 100. Accessing the local database can be useful for times when communication with the infection control facility 148 is unavailable or has been disrupted.

The local database can be used to store private patient information such as the patient's name, social security number. The healthcare facility can send a patient's infection information and medical history data to infection control facility without sending the patient's name and social security number. Only the healthcare facility's local database stores the patient's name and social security number and any other private patient information. This helps to maintain the patient's privacy by refraining from transmitting the patient's private information over the network.

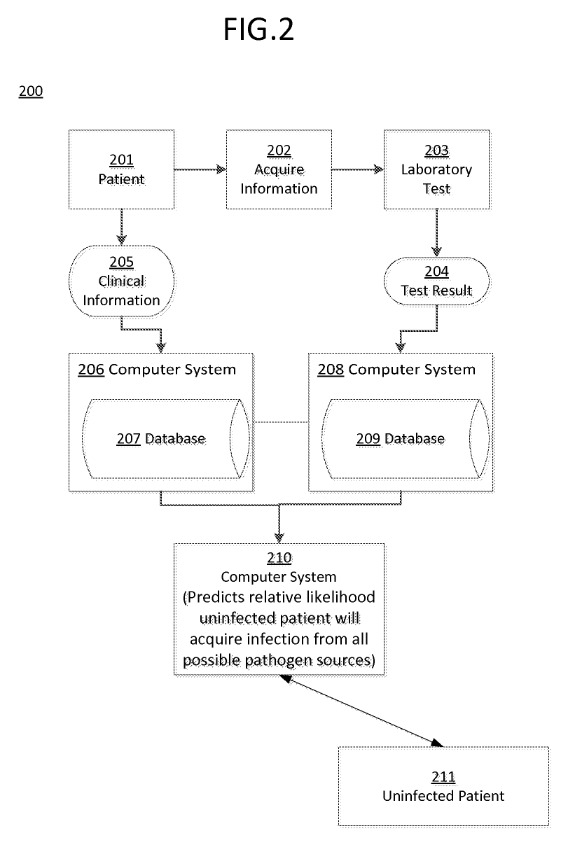

FIG. 2 illustrates an exemplary flow of information 200. When patient 201 in a healthcare facility presents with signs of infection clinical data 205 is collected and entered into a computer system 206 which contains, inter alia, database 207 in the healthcare facility. Information and biological specimens 202 are collected and laboratory tests 203 such as described below are performed. The results of these tests are input into computer system 208 and stored in database 209. Computers 206 and 208 may be the same or different computers. The collected data is transmitted to a computer system 210, which may be as described above in reference to FIG. 1. Computer system 210 analyzes the data as described below and can predict the relative likelihood that an uninfected patient 211 will acquire an infection from a specific pathogen with a specific genotypic or phenotypic profile.

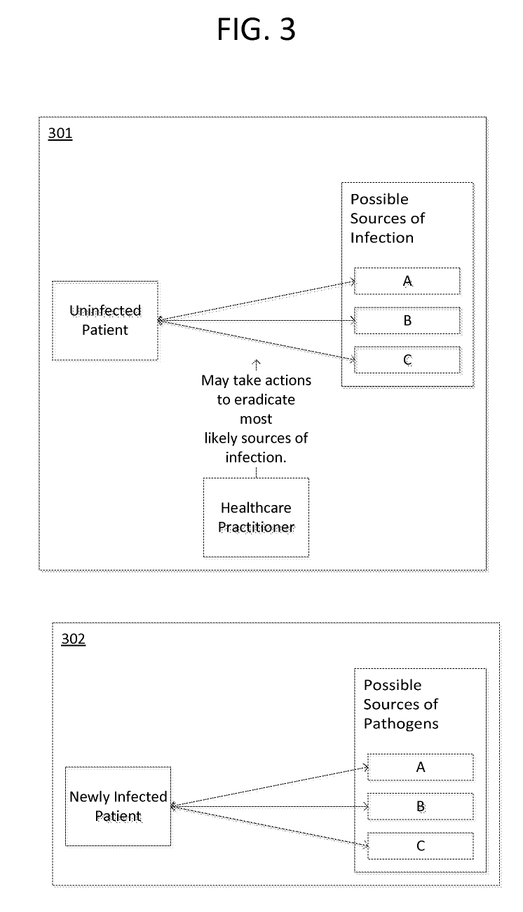

As illustrated in FIG. 3, schema 301 shows that when computer system 210 predicts that an uninfected patient is at risk of infection from possible sources of infection, the system may advise a healthcare practitioner of actions to be taken to eradicate the most likely sources of infection. Likewise, as illustrated in schema 302, when a newly infected patient presents signs of infection, computer system 210 such as server 118 in healthcare facility 148 can compute the possible sources of pathogens by identifying sequential one-away relationships between biological specimens to track the spread of an outbreak to its possible sources. Existing medical techniques can assess whether a patient is more or less likely to acquire an infection by examining risk factors, co-morbidities, etc., and can perform rudimentary analysis to suggest that the person is more likely to get infection from a certain pathogen because there are more of those pathogens locally. However, the systems and methods described herein provide for differentiation among pathogens of the same species according to the particular genotype or phenotype selected for observation. By tracking the source and spread of specific pathogens by genotypic relationships, the likelihood of acquiring an infection of a specific pathogen through a specific vector can be predicted.

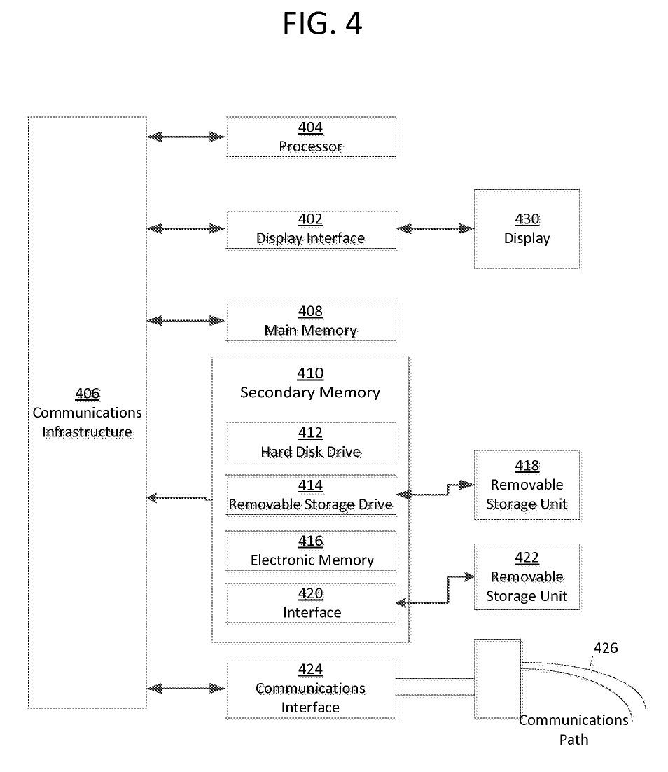

In various embodiments, the server 118 in infection control facility 148, computer systems 160 and 210, and terminals in healthcare facilities 102, 106, 110, and 114 may be as illustrated in FIG. 4. The system contains processor 404, display interface 402, main memory 408, secondary memory 410, and communications interface 424, connected to communications infrastructure 406. A display 430 is connected to the display interface 402. Secondary memory 410 can comprise hard disk drive 412, removable storage drive 414 which is connected to removable storage unit 418, electronic memory, e.g., solid state hard drive, and interface 420 which is connected to removable storage unit 422. Communications interface 424 connects to communications path 426, which may be, for example connected to a network.

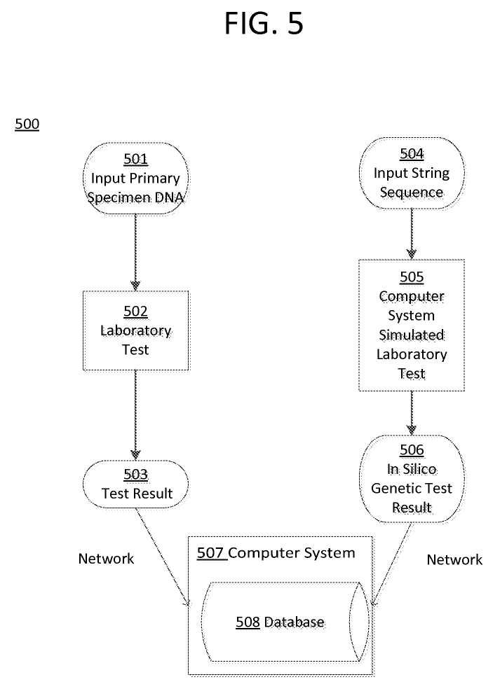

FIG. 5 illustrates data input schema 500 whereby both test results 503 that are produced by a laboratory test 502 conducted on a primary specimen DNA 501 is conveyed, for example via network to computer system 507 and stored in database 508. In silico genetic test results 506, generated as described below in computer simulated laboratory tests 505, can also be transmitted, e.g., via network communications, to computer system 507 to be stored in database 508.

### Cladistics & Microevolution

Cladistics, or phylogenetic systematics, is a system of classification based on the phylogenetic relationships and evolutionary history of groups of organisms, rather than purely on shared features. Modern cladistics analysis assumes:
Any group of organisms are related by descent from a common ancestor.
There is a bifurcating pattern of cladogenesis.
Change in characteristics occurs in lineages over time.

Consistent with these assumptions, microevolution tracks very small changes to a specific population of an organism's lineage regardless of whether those small changes result in changes to observable phenotypic expression.

The methods and systems described herein determine relatedness of closely related entities by recognizing individual state transitions between two distinct states. These systems and methods may be applied in a method of preventing the flow of pathogens in a healthcare facility. In preferred embodiments, when these systems and methods are employed in a healthcare facility, a computer algorithm compares the genotypes and/or phenotypes of a plurality of observed pathogens in order to determine whether two observed pathogens are very closely related.

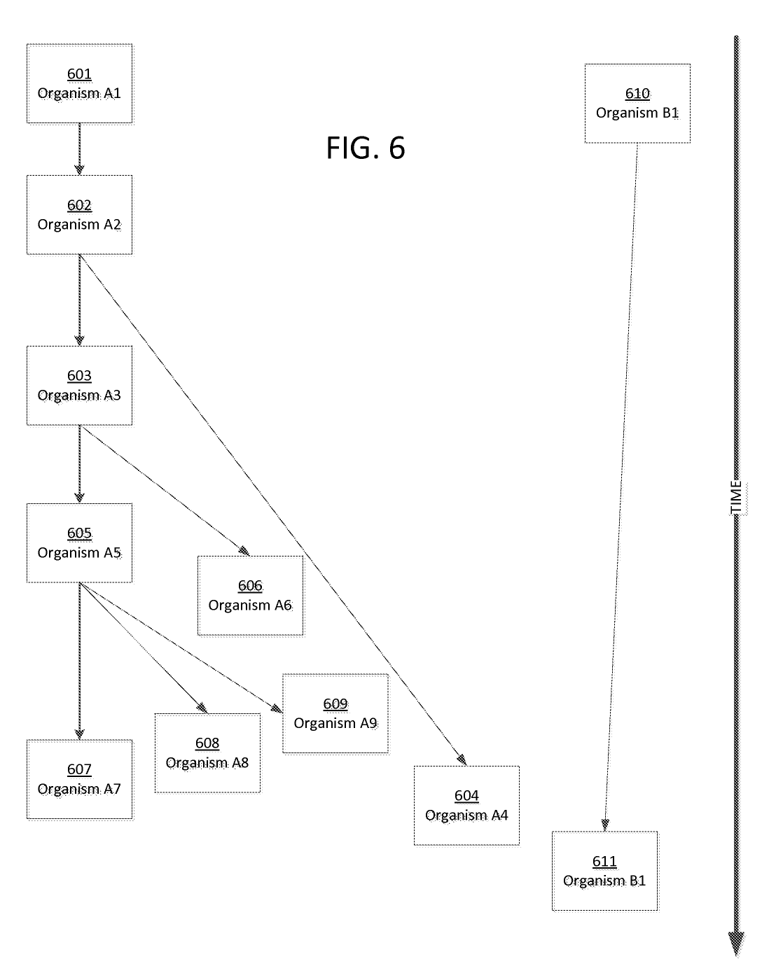

FIG. 6 illustrates relationships between hypothetical closely related organisms. Each directional arrow in this diagram represents a single genetic event. Organism A2 601 is a child of Organism A1 601. Organism A3 603 and Organism A4 604 are children of Organism A2 602. Each child is separated from its parent by one event. However, the event that created Organism A4 604 happened after two additional “generations” of children from Organism A3 603 occurred. Thus, a single genetic event connects Organism A2 602 and Organism A4 604, while Organism A2 602 and Organism A6 606 are separated by two genetic events.

Organism A4 604 is more closely related to Organism A2 602 than Organism A5 605, even though Organism A5 605 and its children Organisms A7, 607, A8 608 and A9 609 were observed before Organism A4 604.

Additionally, a different species of organisms, as indicated in the diagram by Organism B1 610 and Organism B2 611, may mutate at a different intrinsic clock speed. Suppose Organism A mutates at a faster intrinsic clock speed than Organism B. Then, the observation of “N” generational events in Organism A might maintain clonal relatedness between generation 1 and generation N, whereas the observation of “N” generational events in organism B might indicate a completely new clonal cluster because individual events are less common in Organism B than in A.

### Laboratory Tests and the Expression of DNA

A laboratory test has an input and an output. A laboratory test's input may be a primary specimen, a culture consisting of multiple pathogens, isolated DNA or another input format. A laboratory test produces an output that can be analyzed by the human eye or by computer. In a preferred embodiment, a laboratory test is a genetic laboratory test.

DNA sequencing is a laboratory test that accepts isolated DNA as input and outputs a representation of that input DNA as a contiguous string comprised of discrete characters. Other laboratory tests output a phenotypic representation of some, or all, of an organism's DNA. A direct representation of an organism's DNA is called the organism's genotype, whereas an observable characteristic of the organism that results from the composition of an organism's DNA is an example of a phenotype.

Laboratory tests that directly sequence DNA produce a linear string output consisting of one or more discrete characters that represent individual nucleotide molecules. Metaphysically, the result of a DNA sequencing test, the output string sequence, is merely a representation, or “expression”, of the organism's DNA, without actually being the organism's DNA.

Similarly, other laboratory tests express an organism's DNA into other output formats such as a graphic banding pattern, or a series of binary results. For example, a pulse field gel electrophoresis (“PFGE”) laboratory test takes DNA input and outputs a graphic image that consists of a plurality of dark linear bands offset against a light colored background. Another example, the DNA microarray laboratory test takes DNA input and outputs a collection of binary “yes/no” data; yes, if individually queried DNA sequences are found in the original input DNA, or no, if individually queried DNA sequences are not found in the original input DNA. Each of these laboratory tests, and many others, produce equally valid representations of the input DNA sequence. Other examples of laboratory tests that express an input DNA sequence into an analyzable output format include repPCR, MLVA, MLST, etc.

These laboratory tests accept all or some on an organism's DNA sequence as input. Each type of laboratory test expresses DNA with a varying degree of resolution or specificity. For example, direct DNA sequencing resolves individual nucleotide molecules, whereas PFGE tests describe DNA in terms of the measured lengths of smaller DNA fragments that result after the input DNA sequence has been cut into smaller fragments. Although, a PFGE test does not resolve each individual nucleotide molecule, a PFGE test result is a valid representation of an input DNA sequence.

Direct DNA sequencing produces a very accurate representation of an input DNA sequence. However, DNA sequencing is not always practical. Often, other less expensive, faster and more practical laboratory tests that express DNA are used instead of direct DNA sequencing to study organisms' DNA.

Direct DNA sequencing may not even produce the most specific expression of an organism's DNA. A laboratory test can be envisioned that describes the position of a DNA molecule's individual electrons, protons and neutrons. From this output, the composition of DNA nucleotide units could be deduced.

As an analogy, a black and white photograph, a color photograph, a master artist's pencil drawing and a chalk drawing on pavement may all express the face of a living person with varying degrees of specificity and resolution. Similarly, viewing a star in the sky with the human eye, viewing the same star with a hobbyist's telescope and viewing the same star with infrared spectroscopy equipment output different resolutions of the same input target. In Physics, our limited ability to observe and calculate all properties representing a system is called course graining.

The nature of the methods and systems described herein do not care which method is used to express DNA. Any method that can express input DNA into a format that can be analyzed by a human or by a computer is acceptable. The methods and systems described herein embody a system and method to compare a plurality of input DNA regardless of the method of expression. Depending on the methods used, the data input into the system can be comprised of partial or complete nucleotide sequence or expression state information. Complete nucleotide sequence or expression state information can be obtained by whole genome sequencing. Partial nucleotide sequence or expression state information may be obtained by sequencing one or more specifically selected regions of genomic DNA or selected RNA transcripts of genomic DNA, by analysis using a microarray comprising a selection of query sequences, by analyzing restriction enzyme recognition sites in an electrophoretic method, etc.

In preferred embodiments, the system and methods described herein, determine whether the output of two laboratory tests is identical, differs by one genetic event or differs by more than one genetic event. Any laboratory test that expresses an organism's DNA in a manner that can determine whether two expressions of DNA are identical, differ by one genetic event or differ by more than one genetic event may be used as a component of the methods and systems described herein.

### Genetic Events

In modern probability theory, an “event” is a set of outcomes to which a probability can be assigned. An event records the transition from one measurable state to another measurable state. At a moment in time, the state of an organism may be described by:
All of an organism's DNA
A single contiguous subset of the organism's DNA, or
Multiple subsets of an organism's DNA, which may not be contiguous

At a subsequent moment in time, the state of an organism's DNA may have changed, or “mutated”, into a new state that differs from the original state. This DNA mutation event describes a “state transition” from the original state to a new state. At a subsequent moment in time, the new state might remain the same, it might transition back to the original DNA state or it might transition to a new, third state.

Each possible DNA mutation event is described as a genetic event. The methods and systems described herein consider each genetic event to be discrete and to occur at a distinct moment in time, although two genetic events may occur so close together in time that the events cannot be distinguished, and appear to have occurred simultaneously. In preferred embodiments, the systems and methods described herein characterizes a single genetic event to be:
1. A single nucleotide polymorphism, wherein a single nucleotide mutates into another nucleotide.
2. A single nucleotide deletion, wherein a single nucleotide is deleted from string sequence.
3. A single nucleotide insertion, wherein a single nucleotide is inserted into a string sequence.
4. A contiguous nucleotide sequence deletion, wherein one or more contiguous nucleotide sequences, comprising a single unit, are deleted from a DNA sequence
5. A contiguous nucleotide sequence insertion, wherein one or more contiguous nucleotide sequences, comprising a single unit, are inserted into a DNA sequence
6. A contiguous nucleotide sequence reversal, wherein several contiguous nucleotide sequences, comprising a single unit, are reversed at the original position or new position in the DNA sequence.

It will be understood that in the context of DNA, a reverse sequence can refer to the reverse sequence or the reverse complementary sequence.

### Process to Determine Regions of DNA Suitable for One-Away Analysis

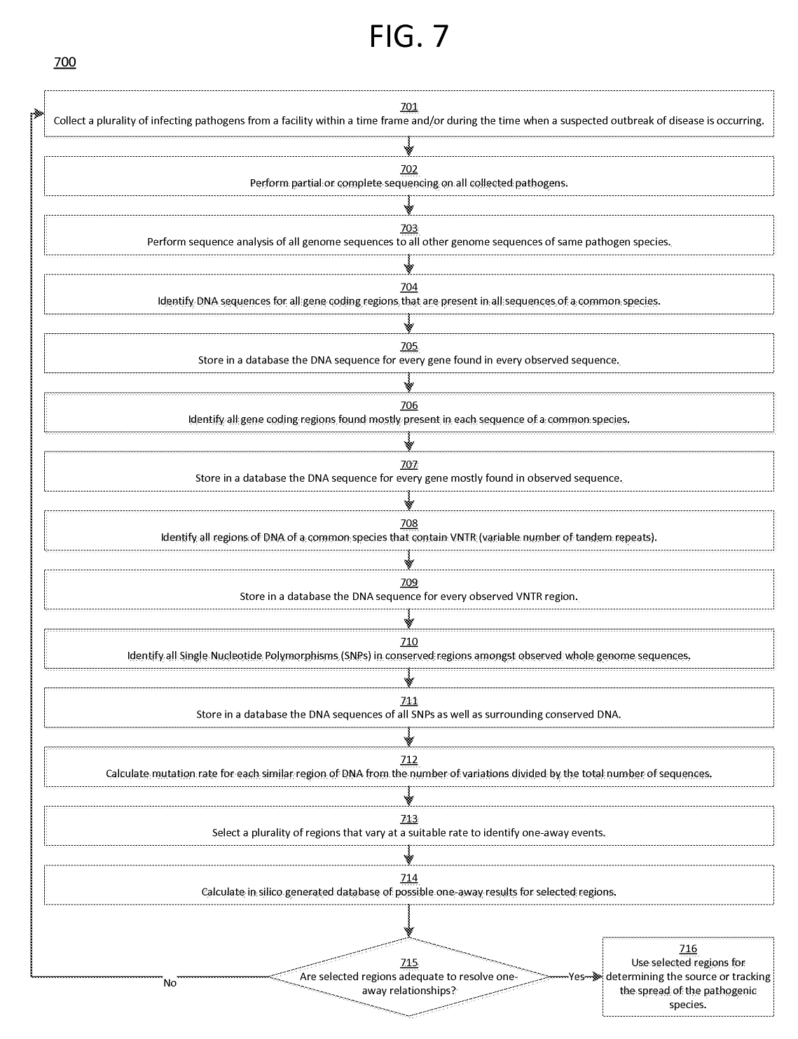

An exemplary process for determining regions of pathogen DNA that are suitable for one-away analysis is illustrated in FIG. 7, which may include the following: At each facility, collect a plurality of infecting pathogens from a facility within a time frame (one month) and/or during the time when a suspected outbreak of disease is occurring. 701 Perform DNA sequencing on all collected pathogens, which may include whole genome sequencing. 702 Perform pairwise sequence analysis of all partial or whole genome sequences to all other partial or whole genome sequences. 703 (Typically, one will compare whole genome sequences to other whole genome sequences and partial genome sequences to any other sequences, including whole genome sequences, in which the sequence being compared has the same regions of DNA. One will only compare genome sequences of the same species.) Identify the DNA sequences for gene coding regions that are present in all sequences of a common species. 704 Store in a database the DNA sequence for every gene found in every observed partial or whole genome sequence. 705 The confirmed presence of a gene in multiple sequences does not mean the DNA sequences for each will be identical in each genome. In fact, variability in conserved genomic coding regions is desired. Identify all gene coding regions found mostly present in each sequences of a common species. 706 Store in a database the DNA sequence for every gene mostly found in observed whole genome sequence. 707 Again, variability in conserved genomic coding regions is desired. Identify all regions of DNA of a common species that contain VNTR (variable number of tandem repeats). 708 Store in a database the DNA sequence for every observed VNTR region. 709 Identify all Single Nucleotide Polymorphisms (SNPs) in conserved regions amongst observed whole genome sequences. 710 Store in a database the DNA sequences of all SNPs as well as surrounding conserved DNA. 711 Thus, four regions of DNA may have been stored in the database as follows:
1) Gene coding regions found in all genomes.
2) Gene coding regions mostly found in all genomes.
3) Regions of DNA containing VNTRs.
4) SNPs.

It is expected that 1) will have the least amount of variability, 2) will have the more variability than 1), 3) will have more variability than 1) and 2), and that the combination of all SNPs identified in 4) will have the most variability.

Compare similar regions of DNA and query for variability amongst sequences originating from different source pathogens. Calculate mutation rate for each similar region of DNA from the number of variations divided by the total number of sequences. 712 Determine the number of identical sequences observed from comparable regions of DNA, and determine the number of variations among comparable regions of DNA. The number of variations for a region divided by the total number of sequences is the simple mutation rate. Each region will have its own mutation rate. The mutation rate for each region may change over time so this process may be repeated in the future.

From the analyzed regions, select a plurality of regions that vary at a suitable rate to identify “one-away” events. 713 The rate of variation can be fine-tuned by selecting a plurality of regions, each with its own clock speed. By properly selecting the regions of DNA for future observation, one-away events can be observed without observing hyper-variability where every sequence appears to be unique. Every facility may host a unique range of pathogens that differs from other similar facilities. Each facility's spectra of pathogens may have its own mutation rates. The regions of DNA selected for molecular typing at one facility may not be optimal for use at another facility. This process of choosing regions of DNA that are suitable for one-away analysis can be conducted for each unique facility, or the regions of DNA that are determined to be most commonly used to discriminate among strains can be applied to other facilities in the same general geographic area. If certain pathogen clones become endemic in a facility, it may be necessary to select new regions of DNA to properly discriminate among strains. Endemic strains may show less variability in the previously identified regions of DNA because the clones are all closely related. When this happens this process is repeated and new DNA target regions are identified.

Once regions have been selected a database of in silico generated one-away results can be generated 714. Historical data sets of test results can be used to determine if the selected regions are adequate to resolve one-away relationships between pathogen samples. These regions can then be used in the methods and systems described herein for determining the source or for tracking the spread of the pathogenic species 716.

### Collection and Analysis of Primary Samples

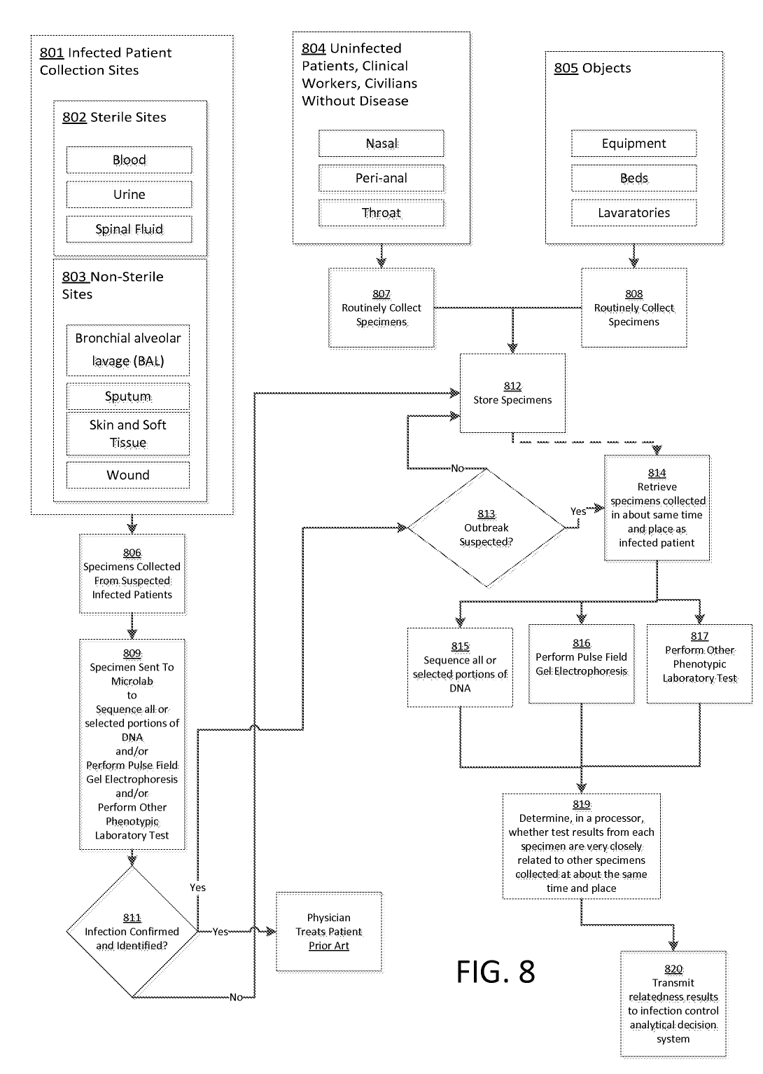

FIG. 8 illustrates the collection and use of biological samples in the systems and methods described herein. Samples can be collected 806 from a variety of sources for different purposes. When a patient presents with signs of infection 801, biological specimens can be selected from sites that are normally sterile 802, e.g. blood, urine, and spinal fluid. Specimens will generally also be collected from sites that are typically non-sterile 803, e.g. bronchial alveolar lavage (BAL), sputum, skin and other soft tissue, and from wounds. These samples are sent to a microbiology lab 809 for confirmation and identification of the sample using one or more methods to confirm and identify the infection, e.g. laboratory tests and phenotypic characterization, sequencing pathogen DNA in whole or in part, and can be used to confirm and identify the infection. If infection is confirmed 811 a physician will treat the infection using standard methods.

In order to track any outbreak through a healthcare facility, and/or identify the source of any outbreak, the facility will also collect 807 specimens from potential sources 804, e.g. un-infected patients at the time of admission, clinical workers, and civilian visitors. The facility can also collect 808 specimens at regular intervals from inanimate objects 805, e.g. equipment, beds, and laboratories. These specimens can be stored 812 for later use in the event that an outbreak is suspected. If an outbreak is suspected, specimens collected at times and in places proximate to the infected patient can be retrieved 814 and tested using laboratory tests and phenotypic characterization 817, sequencing pathogen DNA in whole or in part 815, and pulse field gel electrophoresis 816. The results of these exams are input into a computer system for determination programmed to carry out a one-away analysis to determine whether test results from each specimen are very closely related to other specimens collected at about the same time and place using the methods and systems described herein. 819 The related relatedness determination can be transmitted to an infection control analytical decision system 820 and used to identify the source of the infection and track its spread.

### Interpreting Laboratory Test Results to Infer the Occurrence of a Single Genetic Event

The detection of a single genetic event requires observing two distinct states: a before state and an after state. Therefore, the detection of a single genetic event requires comparing a plurality of laboratory test results, wherein each laboratory test result describes the state of an organism at a moment in time. In preferred embodiments, the systems and methods described herein determine whether two laboratory tests produce results that are:
1. Identical
2. Differ by a single genetic event, or
3. Differ by more than one genetic event

Determining identity is trivial. Identity can be determined if the output results from two distinct laboratory tests appear the same within an accepted margin of error. It should be noted that if two distinct laboratory tests produce identical results, then it does not necessarily mean that the two input DNA sequences used in each distinct test are absolutely identical. The particular type of laboratory test may not have sufficient resolution to determine whether two inputs are exactly identical. Instead, the resolution of the laboratory test may only be able to determine that two input DNA sequences are similar, even though the output results of two laboratory tests are identical. Additionally, two laboratory tests may produce identical results when the input DNA reflects a subset of an organism's entire DNA state. Identical results may only mean that the input DNA sequences are identical. The state of two organisms' entire DNA may differ.

Furthermore, it should be clarified that two identical test results does not mean that a single organism's DNA was input into two separate laboratory tests. Instead, each DNA input may have been collected from two distinct strains that happen to have the same composition of DNA in the region queried.

If a comparison of two distinct laboratory tests results illustrates the occurrence of a single genetic event, then one can describe the two input DNA as being “one-away” from the other, or “one genetic event away” from the other. Comparison of a plurality of laboratory test results can be used to build a visual graph that displays phylogenetic relatedness.

### DNA Sequencing

DNA sequencing is a laboratory test that expresses input DNA as an output linear string comprised of discrete letter characters that represent individual nucleotide molecules. There are several DNA sequencing technologies that express input DNA as an output string value. DNA sequencing can be performed on one or more regions of an organism's DNA. The output string sequences can be analyzed individually, concatenated with other output strings or combined into one or more consensus sequences wherein regions of DNA that may have been sequenced multiple times are accounted for and not counted multiple times. DNA sequencing is the current standard against which other tests that express DNA are compared.

### Determining Relatedness Between Two DNA Sequences

A DNA sequencer accepts isolated DNA as input and outputs a string sequence. Two DNA inputs can be compared by comparing the two output string sequences to see if the two string sequences are:
1. Identical.
2. Differ by exactly one event, i.e. are “one-aways.”
3. Differ by more than one event, or are more than one-away.

For this algorithm, the string used as input into the algorithm may represent a contiguous region of DNA collected at a single locus, or the string used as input into the algorithm may represent a plurality of DNA collected from a plurality of loci that have been concatenated into a single input string sequence.

A computer system programmed to compare sequences of characters can trivially determine whether two string sequences are identical. The systems and methods described herein provide an improved algorithm to determine whether two string sequences are “one-away” from the other.

The “Edit Distance Algorithm” is a classic computer science algorithm that is used to determine how closely two strings resemble each other. Edit distance, also referred to as “Levenshtein distance,” is the minimum number of character insertions, deletions, and substitutions needed to transform one string to the other. Edit distance and its weighted variants, where edit operations are associated with different positive costs, are important primitives with numerous applications in areas such as computational biology and genomics, text processing, and web searching. Many of these practical applications typically deal with large amounts of data ranging from a moderate number of extremely long strings, as in computational biology, to a large number of moderately long strings, as in text processing and web searching. Therefore methodologies for edit distance that are efficient in terms of computational resources (running time and/or storage space), even with modest approximation guarantees, are highly desirable. See, for example, US Patent Application Publication 2007/0085716, incorporated herein in its entirety.

Edit distance algorithms have been extensively studied. Traditional edit distance algorithms employ dynamic programming methods that calculate minimum edit distances by recursively subdividing the problem domain into smaller problem domains and first finding optimal solutions to the smaller problem domain. Dynamic programming methods usually result in several optimal solutions. Traditional dynamic programming methodology computes edit distance in quadratic time and the methodology can be made to run in linear space. The quadratic time methodology for computing the edit distance has generally improved by only a logarithmic factor, and even developing sub-quadratic time methodologies for approximating it within a modest factor has proved to be generally challenging. Current algorithm design has focused on finding faster solutions to an approximate edit distance solution.

There are many variations of the classic edit distance algorithm. These include the Needleman-Wunsch algorithm, the Smith-Waterman algorithm and other weighted edit distance algorithms. These algorithms may be applied to any string input but are often applied to biologic sequence data such as nucleotide or amino acid protein sequences.

In the realm of computational biology, scientists employ these algorithms to determine how closely related nucleotide or protein sequences are and, with this measure of relatedness, build phylogenetic trees that visually display degrees of relatedness among multiple organisms.

The edit distance algorithm and its variants have proven useful to infer relatedness but have also shown a weakness when analyzing large quantities of string data because of its mostly quadratic running time. In preferred embodiments, an element of the system and methods described herein is a unique algorithm that determines whether two input strings differ by a single one-away event.

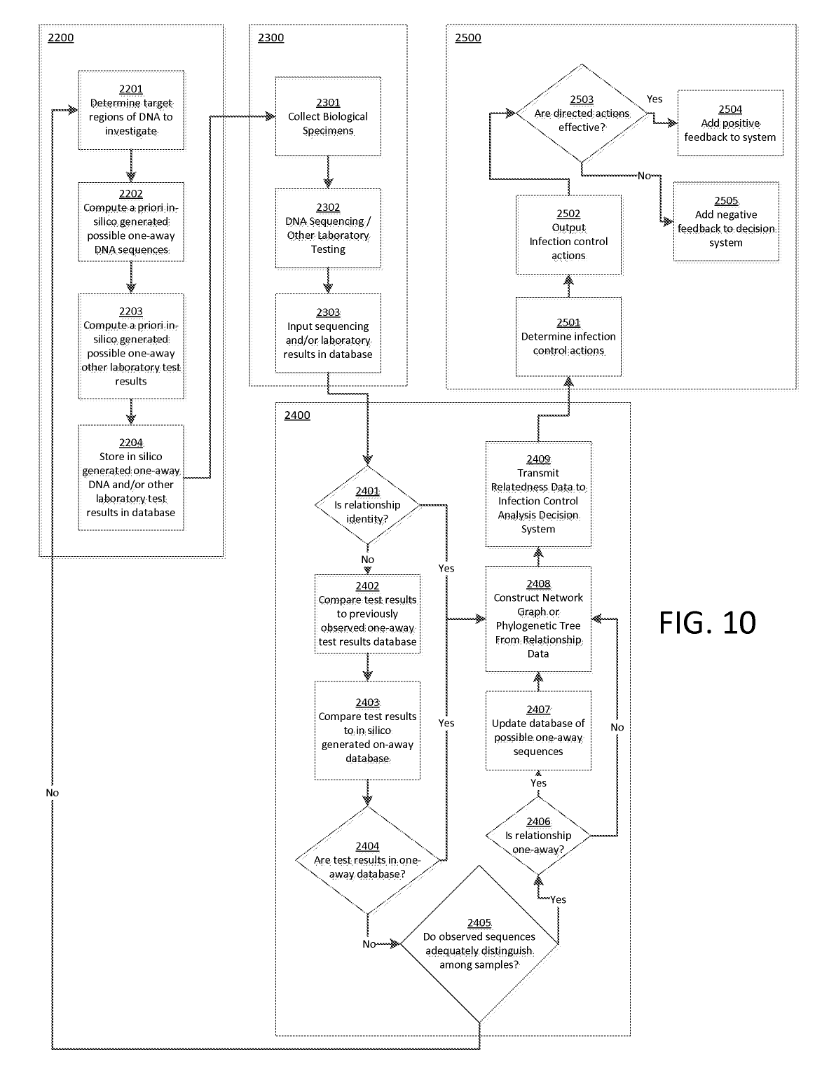

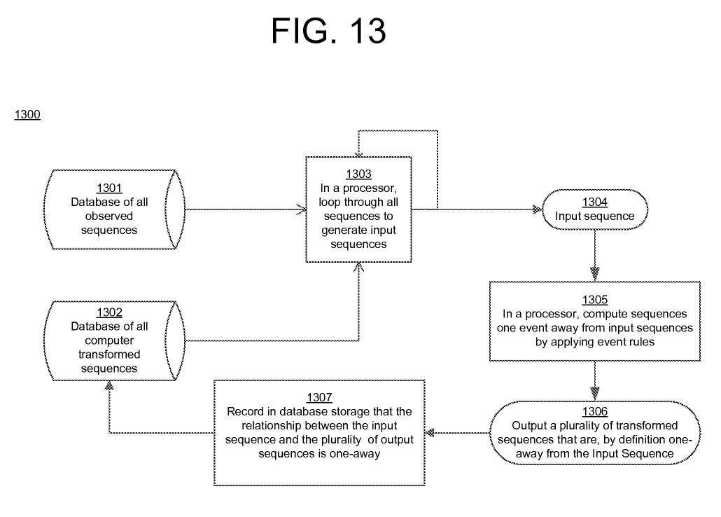

An exemplary application of the system and methods described herein is illustrated in FIG. 10. The system is initialized using a procedure 2200 comprising determining target regions of DNA to be investigated 2201 for example using the procedure 700 described above. A database of in silico generated possible sequences that are one-genetic event away from each other (a one-away database) can be generated 2202, for example using the procedure 1300 illustrated in FIG. 13. This database can be used to compute, in a processor, a database of possible one-away laboratory test results 2203 which are stored for later reference. Biological samples are then collected and test results obtained. 2300, 2301, 2302. Sequencing and/or other laboratory test results are stored in the system database. 2303. When an infection occurs, or an outbreak is suspected, these DNA sequencing results can be retrieved and analyzed to determine the relationship between two specimens 2400. If two samples are the same, the identity relationship is transmitted to an infection control analysis decision system. 2408 If the relationship is not identity, the test results are compared to all previously recorded one-away test results in the database 2402 and to the in silico database of one-away test results. If the results are found in the database, then the one-away relationship is transmitted to an infection control analysis decision system 2408 which can build a network graph or phylogenetic tree to track the pathogen and/or identify its source. If the results are not found in the database, the system determines whether the observed sequences are adequate to distinguish among samples 2405. If the answer is negative, the system can be refined by repeating the initialization procedure 2200 based he observed sequences. If the answer is positive, the pairwise relationship is determined 2406. If the relationship is determined to be one-away, the one away database is updated 2407. The relationships that have been determined are then used to construct a network graph or phylogenetic tree 2408. The graph or tree is generally built from one-away relationships taking into consideration time and place data for each sample. However, same or more than one-away relationships are also relevant. The relationship data, e.g. the network graph, is transmitted 2409 to an infection control analysis decision system 2500.

The infection control analysis decision system 2500 receives the relationship data and can determine 2501 and output 2502 recommended infection control actions. The effectiveness of the actions are determined 2503. If the actions are effective, the system is updated with positive feedback 2504. If the actions are not effective, the system is updated with negative feedback 2505.

### DNA Sequence One-Away Algorithm

The one-away string algorithm determines whether two string sequences are identical, differ by one event or differ by more than one event. The algorithm runs significantly faster than the quadratic edit distance algorithm. Additionally, the result of every string comparison is recorded in a database so that future analysis can first be compared to a cached look-up of previously recorded comparisons.

When comparing two input strings, the algorithm abandons analysis as soon as the algorithm determines that two input strings are more than one-away from the other. Thus, the running time of the algorithm is significantly better than quadratic.

The one-away algorithm stores the output relationship between all previously analyzed input strings in a “database”. The database allows the output of future string comparison to be looked up in a cached look-up list in order to possibly avoid computationally expensive further analysis.

### The Sequence One-Away Algorithm

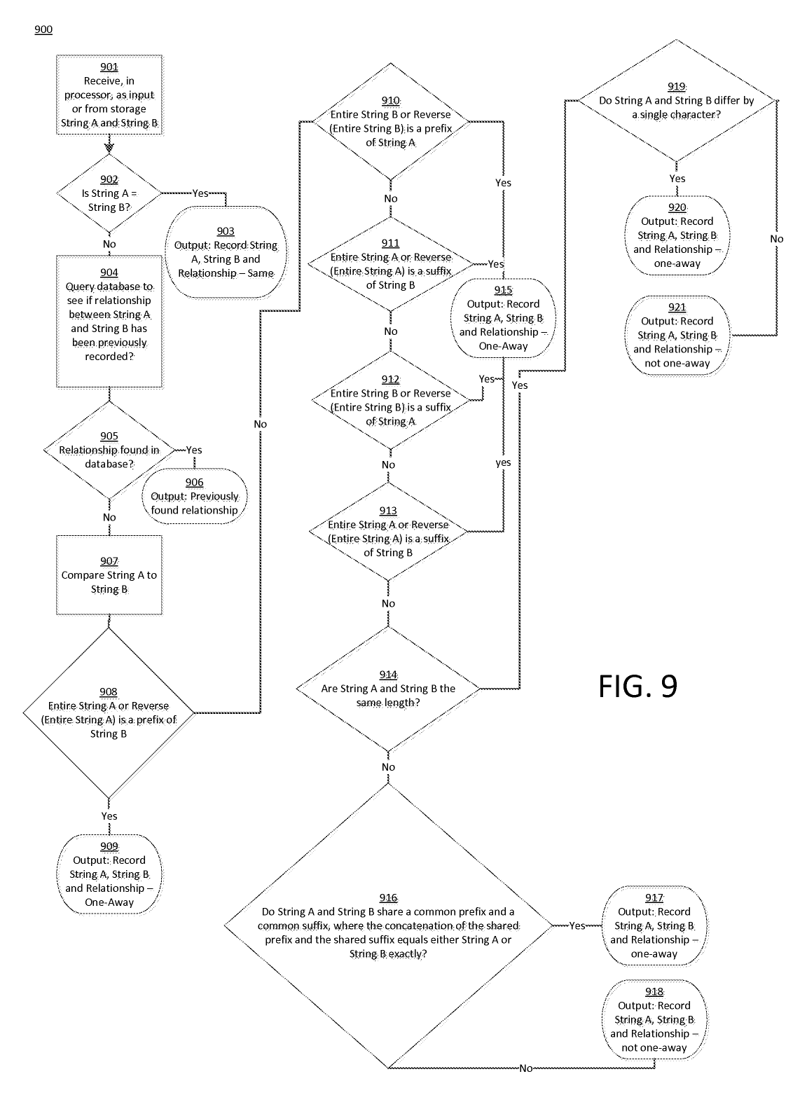

A sequence one-away algorithm 900 may comprise the following steps as illustrated in an exemplary embodiment in FIG. 9:
1) Two string sequence inputs, String A and String B can be received in a processor 901, either as user input, or retrieved from storage, e.g. from a database stored on a hard drive.
2) If String A is identical to String B 902, output that the two strings are identical and exit the algorithm. 903
3) Search “database” to see if the relationship between String A and String B has been previously recorded as being “one-away” or “more than one-away” from the other. 904 If the relationship between String A and String B has been recorded, output the cached relationship and exit the algorithm. 905
4) The strings are compared to each other. 907 Check to see if String A in its entirety is a prefix of String B. 908 If String A is a prefix of String B, then String A and String B are separated by one genetic event. Record the relationship as “one-away” in the database, output “one away” and exit algorithm. 909
5) Check to see if String B in its entirety is a prefix of String A. 910 If String B is a prefix of String A, then String A and String B are separated by one genetic event. Record the relationship as “one-away” in the database, output “one away” and exit algorithm. 915
6) Check to see if String A in its entirety is a suffix of String B. 912 If String A is a suffix of String B, then String A and String B are separated by one genetic event. Record the relationship as “one-away” in the database, output “one away” and exit algorithm. 915
7) Check to see if String B in its entirety is a suffix of String A 913. If String B is a suffix of String A, then String A and String B are separated by one genetic event. Record the relationship as “one-away” in the database, output “one away” and exit algorithm. 915
8) If String A and String B are the same length 914, check to see if they differ by exactly 1 unit difference 919. A unit may be a single character 919 or a contiguous concatenation of characters 916. As soon as it is determined that String A and String B differ by two units, then record the relationship as “more than one-away” in the database, output “more than one away” and exit algorithm. 921 If String A and String B are the same length and they differ by exactly by one unit, then record the relationship as “one-away” in the database, output “one away” and exit algorithm 920.
9) If String A and String B have different lengths 914, and if String A and String B share a common prefix and if String A and String B share a common suffix and if the concatenation of the common prefix and the common suffix exactly equals either String A and String B then record the relationship in the database and output “one genetic event away” 917, otherwise record the relationship and output “not one genetic event away” and exit algorithm 918.
   Additional rules can further refine the results of the algorithm. For instance, in step 8, the rule recognizes the insertion of a contiguous string element into an original string. This can be made more specific by determining if certain specific types of strings are inserted into the originating string. For example, a further refining rule might be an exact copy of the “n” characters that precede a specific nucleotide, or the “m” characters that follow a particular may be inserted into the sequence. So, not only is a contiguous string inserted, but it is a specific string—the copy of a sequence, or reversal of a sequence that already exists in the originating sequence. Another rule might determine if specific genetic events occur at certain positions. Such rules can indicate the direction of time because the event may only occur in one direction. For example, a specific event rule might be any 10 characters can be inserted, which is a more specific one-away event, and another rule might be only 3 characters can be deleted. However, because the events are asymmetric it may be possible to transition from sequence A to Sequence B but not from Sequence B to Sequence A. Applying such logic can help determine which strains appeared first in time.
   Inferring Single Genetic Events from Laboratory Tests Other than DNA Sequencing

One-away algorithms that are similar to the previously described string one-away algorithm can be implemented for laboratory tests other than DNA sequencing. If the method by which a laboratory test produces its output format from input DNA is well understood, then that laboratory test can be simulated on a computer. Such computer simulations are described as “in silico” experiments because the laboratory test is “performed” on a computer. The in silico experiment accepts a string sequence, that represents actual DNA, as input. The in silico experiment generates a simulated output format based on knowledge of how the actual laboratory test expresses actual DNA input into actual output. The output of an in silico experiment should match exactly the output of the corresponding physical laboratory test.

In order to develop an in silico algorithm, the means by which the laboratory test expresses DNA should be well understood. Examples of specific one-away algorithms are presented below. However, it should be noted that the methods and systems described herein work for any laboratory test that expresses DNA.

Inferring Single Genetic Events from PFGE Tests

Pulsed Field Gel Electrophoresis (“PFGE”) is an example of a laboratory test that expresses input DNA as a graphic image that consists of a plurality of dark bands arranged in a linear pattern against a light background. Other examples of laboratory tests that express an organism's DNA as a banding pattern include MLEE, repPCR, and ribotyping.

Laboratory tests that express DNA as an image-based banding pattern are not able to resolve individual nucleotide molecules. However, the comparison of two image-based banding patterns may identify single genetic events.

The PFGE test uses a restriction enzyme to cleave input DNA sequence into multiple, smaller fragments. The resulting shorter DNA fragments are sorted according to fragment length. The sorted fragments are stained to visually highlight resulting fragments as a band. Each visual band represents the length, or more accurately the molecular weight, of each resulting DNA fragment.

Restriction enzymes recognize specific patterns of nucleotide sequences and cut a linear DNA strand into two pieces at each recognition site. A DNA strand that has multiple recognition sites will be cleaved into multiple segments. Two DNA sequences that have a different number of restriction sites, or two sequences that have a different number of nucleotide sequences between two common restriction sites, will produce different PFGE banding test results.

Different restriction enzymes recognize different nucleotide patterns. Any restriction enzyme may be used to perform a PFGE test but typically a restriction enzyme is selected so that input DNA will be cleaved at multiple restriction sites. Additionally, restriction enzymes are selected so that not too many bands appear in the output result so that the results can be easily interpreted by the human eye.

After a DNA sequence has been cleaved at each restriction site, the original single strand of DNA transforms into multiple shorter DNA sequences, which, if reassembled in a correct order, would result in the original DNA sequence. The resulting smaller strands of DNA are placed into an agarose gel where a varying electric field pushes the cleaved DNA sequences through the gel. The final resting point of each DNA segment depends on the length of each segment. The distance travelled through the agaraose which is proportional to its molecular weight which corresponds to the DNA segments aggregate electrical charge. If two strands of DNA have the same length then they will appear in approximately the same band in the PFGE banding pattern. After each DNA segment arrives at its final resting place, the gel is stained. The stain illuminates the final resting position of each DNA segment. Typically, each PFGE test is compared to the banding pattern produced by a known reference sequence whose bands correspond to a known molecular weight.

Similar to the DNA sequencing laboratory test result, PFGE test results can be compared in order to determine if the results are identical, differ by one genetic event (a one-away relationship) or differ by more than one genetic event (more than one-away relationship).

### Interpreting PFGE Test Results

In order to determine whether two PFGE laboratory test results differ by a single genetic event, it helps to understand how changes in an organism's DNA affect the corresponding PFGE test result. A PFGE banding pattern changes when one of the following genetic events occur:
1. A SNP occurs at the site of an existing restriction enzyme recognition site, thereby eliminating the restriction enzyme pattern and combining two previously cleaved DNA strands into one “uncleaved” strand of DNA.
2. A SNP occurs resulting in the addition in a new restriction enzyme recognition site, thereby cleaving a larger strand of DNA into two.
3. A contiguous region of multiple nucleotide sequences is inserted between two existing restriction sites, and that contiguous region does not include any restriction enzyme recognition sites, thereby increasing the length of the existing DNA sequence located between two restriction enzyme patterns.
4. A contiguous region of multiple nucleotide sequences is inserted between two existing restriction sites, and that contiguous region includes one or more restriction enzyme recognition sites, thereby increasing the number of cleaved DNA fragments and increasing the number of bands in the output banding pattern.
5. A contiguous region of multiple nucleotide sequences is deleted between two existing restriction sites, and that contiguous deleted region does not contain any restriction enzyme sites, thereby decreasing the length of the existing DNA sequence located between two restriction enzyme patterns.
6. A contiguous region of multiple nucleotide sequences is deleted between two existing restriction sites, and that contiguous deleted region does contain one or more restriction enzyme sites, thereby decreasing the number of resulting cleaved fragments and decreasing the number of bands in the output banding pattern.

The comparison between other laboratory tests that express DNA as an image of a linear banding pattern may be interpreted in a similar manner.

### PFGE One-Away Algorithm

Because PFGE does not resolve DNA as well as DNA sequencing, it may not be possible to absolutely determine whether two PFGE test results differ by single genetic event. Instead it is easier to determine whether two banding patterns are identical, or whether two banding patterns are more than one event away from the other.
1. If a SNP occurs at the site of an existing restriction enzyme recognition site, thereby eliminating the recognition site, then two smaller electrophoretic bands will disappear from the original banding pattern and reappear as a single larger band. The molecular weight of the larger band will equal the sum of the molecular weights of the two smaller bands. All other bands will remain in the same position.
2. If a SNP occurs that results in the addition in a new restriction enzyme recognition site, thereby cleaving a strand of DNA into two fragments, then a single “heavier” electrophoretic band will disappear from the banding pattern and be replaced by two “lighter” bands. The sum of the molecular weights of the two lighter bands should equal the molecular weight of the original heavier band. All other bands shall remain in the same position.
3. If a contiguous region of multiple nucleotide sequences is inserted between two existing restriction sites, and that contiguous region does not include any restriction enzyme recognition sites, thereby increasing the length of the existing DNA sequence located between two restriction enzyme patterns then a single band will “move” in the banding pattern from representing a lighter weight strand of DNA to representing a heavier strand of DNA. The delta between the molecular weight of the original band and the new band shall represent the molecular weight of the inserted DNA sequence. All other bands shall remain in the same position.
4. If a contiguous region of multiple nucleotide sequences is deleted between two existing restriction sites, and that contiguous deleted region does not contain any restriction enzyme sites, thereby decreasing the length of the existing DNA sequence located between two restriction enzyme patterns, then a single band will “move” in the banding pattern from representing a heavier of DNA to representing a lighter strand of DNA. The delta between the molecular weight of the original band and the new band shall represent the molecular weight of the deleted DNA sequence. All other bands shall remain in the same position.
5. If a contiguous region of multiple nucleotide sequences is inserted between two insertion sites (or between the origin and end location of the original DNA sequence if there no restriction enzyme recognition sites existed in the original sequence) and the contiguous inserted region contains one or more restriction recognition sites thereby resulting in additional cleaved DNA sequences, then it may not be possible to recognize this event as a single genetic event by solely examining the resulting electrophoretic banding pattern. However, in this scenario, the organism's entire genome, or, certain specified regions of the organism's DNA can be DNA sequenced and compared “in silico” to the actual electrophoretic banding pattern to determine whether a single genetic event caused the change in banding pattern.
6. If a contiguous region of multiple nucleotide sequences is deleted from a DNA sequence and that deleted DNA sequence contains one or more restriction enzyme recognition sites thereby resulting in fewer cleaved DNA sequences, then it may not be possible to recognize this event as a single genetic event by solely examining the resulting electrophoretic banding pattern. Again, the organism's entire genome, or, certain specified regions of the organism's DNA can be DNA sequenced and compared “in silico” to the actual electrophoretic banding pattern to determine whether a single genetic event caused the change in banding pattern.
7. If a contiguous region of DNA is inserted into a DNA sequence in the middle of a restriction enzyme site, or a contiguous region of DNA is deleted from a DNA sequence and the deleted region of DNA contains some, but not all of a restriction enzyme recognition site, then it may not be possible to recognize this as a single genetic event by solely examining the resulting electrophoretic banding pattern. In a similar manner to 6 and 7 above, a genome can be sequenced and compared in silico to the actual electrophoretic banding pattern to determine whether a single genetic event occurred.

By comparing electrophoretic banding patterns that result from two organism's DNA, the outputs can be identified as being “identical”, “one genetic event away” or “more than one genetic event away” even though the PFGE test results are not able to resolve the state of the organism's DNA as well as the DNA sequencing laboratory test.

### In Silico PGFE Experiments

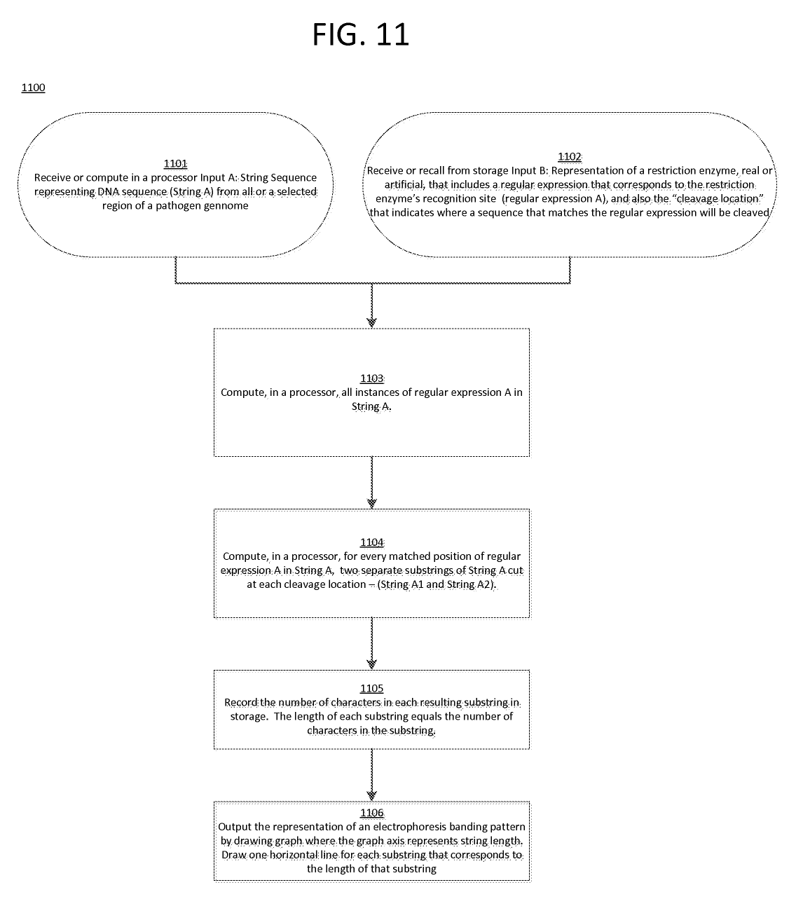

PFGE laboratory tests can be simulated in silico. An exemplary algorithm for generating a PFGE test result in silico 1100 is illustrated in FIG. 11. An input string (String A) representing a DNA sequence is received in a processor, or may be computed by transforming a string by an event rule 1101. A representation of a restriction enzyme in the form of a regular expression corresponding to the enzyme's recognition site and the cleavage location where the enzyme cuts DNA in relation to the recognition site is received in the processor as input or recalled from a database of restriction enzyme data 1102. Given an input string sequence, the algorithm can discover the location of all restriction enzyme recognition sites, “cut” the input sequence at those points, count the number of characters in each resulting string fragment and plot the resulting fragment sizes. All instances of the regular expression in String A are computed in the processor 1103. For every matched position of regular expression A in String A, two separate substrings of String A cut at each cleavage location (String A1 and String A2) are computed 1104. The number of characters in each substring are recorded 1105. If the input String A represents circular DNA, the original string has no endpoints, whereas a singly cut String A will not have substrings, but rather will be a linear DNA of the same number of characters, and this result is recorded. An output representation of an electrophoresis banding pattern can be drawn 1106 where the graph axis represents string length, drawing one line for each substring that corresponds to the length of that substring. The resulting output should resemble the image output of an actual PFGE laboratory test conducted with real restriction enzymes cutting real DNA.

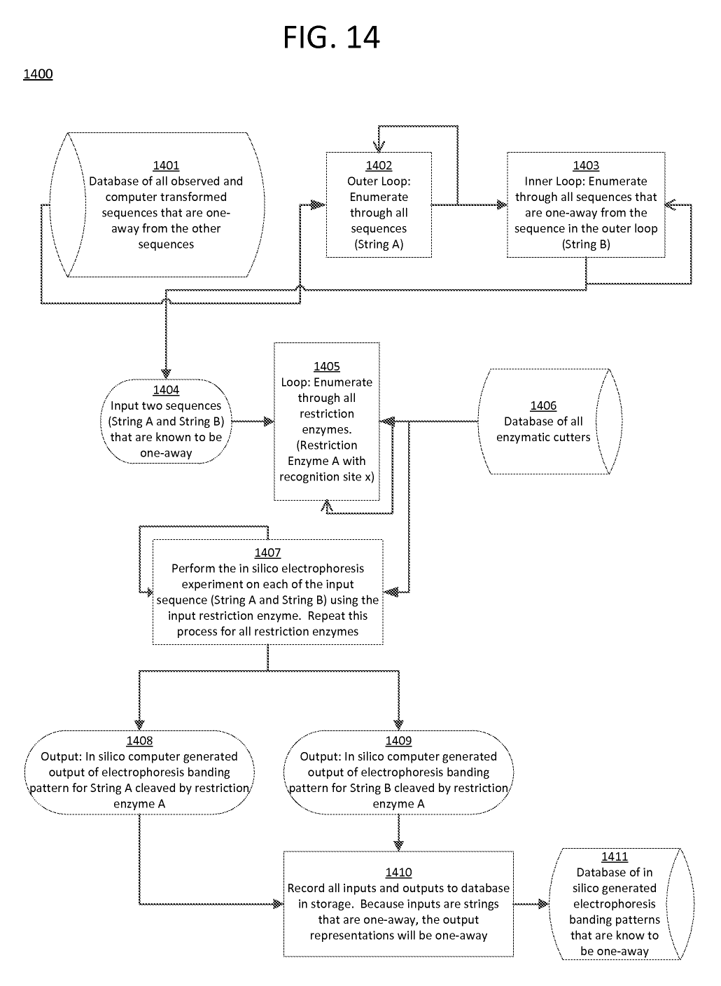

In silico PFGE tests can be performed on one or more known DNA sequences using all known restriction enzymes. An exemplary algorithm 1400 is illustrated in FIG. 14. A database of observed and computer transformed sequences that are one-away from the other sequences is input 1401. In an outer loop, the algorithm enumerates each sequence to input 1402 In an inner loop, each sequence that is one genetic event away from String A is enumerated (one-away) 1403. The sequences may include all possible sequences that are one away even though many of these transformations will not produce an observable change in PGFE test results, or one may use sequence transformation rules based on an understanding of the types of sequence transformations that may produce a different PGFE result as described above to generate, in silico, a listing of only those one-away DNA that will result in an observable difference in a PGFE test result. Each pair of one-away sequences String A and String B are taken as input 1404. The processor then enumerates 1405 each restriction enzyme in a database of all suitable enzymatic cutters 1406. The algorithm 1100 for generating an in silico PFGE test result is then performed repeatedly for each String A, String B and enzyme 1407. The electrophoresis banding patterns for String A and String B are output 1408, 1409 and can be recorded 1410 to generate a database of in silico generated banding patterns that are known to be one-away 1411.

Additionally, a computer can alter a given input DNA sequence by one genetic event, for example using the algorithm 1300 illustrated in FIG. 13 and then conduct the same in silico PFGE test. This method can be used to build a database of sequences that are known to be one-genetic event away from each other. A database of observed sequences 1301 is input into a processor, which loops through the sequences 1303 to generate input sequences 1304. The processor then computes 1305 sequences that are one-away from the input sequence using a list of potential genetic event rules 1306 which are recorded 1307 into a database 1302 which can then be used recursively as inputs into the algorithm. When the results are used as inputs for generating in silico PFGE results, the resulting output represents a database of theoretically possible one-away PFGE test results. This process can be repeated ad infinitum.

Furthermore, as additional actual DNA sequences are obtained and analyzed using actual laboratory tests, observed genetic events can be hypothetically applied to all other previously observed DNA sequences in order to build a catalog of all observed in silico PFGE results. Observed PFGE test results can be compared to theoretical in silico test results to assist in the determination of whether two PFGE test results are one-away from each other. Applications of in silico test results are described in greater detail below.

### DNA Microarray Test

DNA microarray tests query whether certain single nucleotide polymorphisms (“SNPs”) exist in input DNA. Microarray tests identify, thousands, if not millions, of SNP's in one output result. A DNA microarray test does not identify each and every nucleotide molecule in an input DNA sequence. Instead, a DNA microarray test reports whether a particular queried SNP exists or does not exist in input DNA. Therefore, a DNA microarray test expresses DNA as a plurality of binary yes/no results that describe the presence or absence of SNPs in the input DNA.

DNA microarray tests may be designed to query input DNA for the presence of SNPs that are known to exist only in certain contiguous regions of DNA such as a gene, a pathogenicity island, or other insertion element. Thus, by querying input DNA for a particular SNP, the DNA Microarray test may learn whether an entire gene, pathogenicity island, or other contiguous region of DNA is present or absent in the input DNA.

The output results from two DNA microarray tests can be compared to determine whether the test results are identical, differ by one genetic event (a one-away relationship), or differ by more than one genetic event. Actual DNA sequencing laboratory test may be used to output binary yes/no answers if the resulting DNA sequences are queried for the presence or absence of specific sequences.

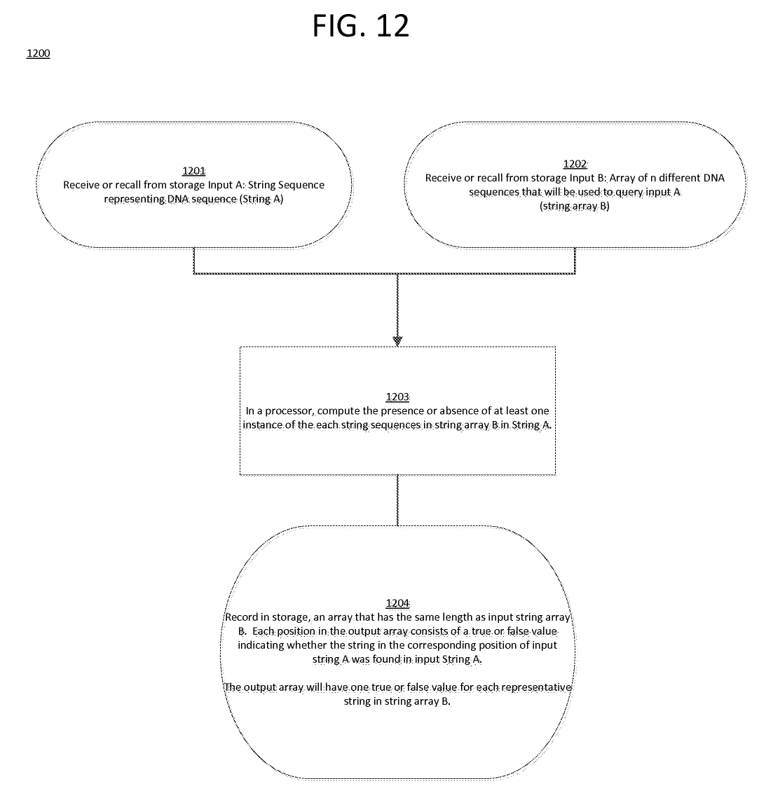

A database of in silico test results can be generated, for example using the algorithm 1200 illustrated in FIG. 12. An input String A is received in a processor or recalled from storage. 1201 An array of DNA sequences Array B is input or recalled into the processor 1202 which then computes the presence or absence of each string sequence in string array B in String A. 1203 The results are recorded in storage 1204 to build a database of in silico test results. Each position in the output array consists of a true or false value indicating whether the string in the corresponding position of input string A was found in input String A. The output array will have one true or false value for each representative string in string array B.

### Interpreting DNA Microarray Test for One Away Events

Determining identity between two DNA microarray test outputs is trivial. If two DNA Microarray test results produce identical results except for one single binary answer, then those two test results are “one away.” Other laboratory tests that produce a collection of binary outputs can be interpreted in a similar manner.

### Microarray One-Away Algorithm

A microarray one-away algorithm may comprise the following steps:
1) If all the binary outputs of Microarray Test 1 are the same as Microarray Test 2, output that the two tests are identical and exit the algorithm.
2) If all binary outputs of Microarray Test 1 have two (or more) differences from the binary outputs of Microarray Test 2, output that the two tests are “not one genetic event away” and exit the algorithm.
3) If all binary outputs of Microarray Test 1 have exactly one difference from the binary outputs of Microarray Test 2, output that the two tests are “one genetic event away” and exit the algorithm.

### Combining Actual Laboratory Results and in Silico Results

A laboratory test that expresses DNA can be simulated using computer software that accepts a known DNA sequence as input. The two can be differentiated as a laboratory test result and an in silico test result. Both a laboratory test result and an in silico test result can be stored in a database.

One-away relationships between two test results can be stored in a database, for example a one-away relationship between two laboratory test results can be stored in a database. Storing the relationship between two laboratory test results in a database may allow relationships between other test results to be “looked up” without having to compare and compute the differences between actual laboratory test results.

Relationships that can be stored in the database include:
1) Result A is one event away from Result B, or
2) Result A is not one event away from Result B.

There would be no need to consult the database if Result A and Result B are identical. When new laboratory results are observed, the new laboratory results can be compared to any or all previously observed laboratory results that have been saved in the database to determine if the new result is “one-away” from each previously observed laboratory result.

Laboratory test results can be also be compared to previously computer-generated in silico test results. In silico test results can be generated by varying the input DNA sequences and storing the output results. In silico test results can be produced without having conducted an actual corresponding laboratory test. Therefore, two previously unobserved laboratory test results could be compared to previously generated in silico test results. If both laboratory test results match previously generated in silico test results, the relationship between the laboratory test results can be rapidly determined by noting the previously analyzed one-away relationship between the matching in silico test results.

### Automatically Generating in Silico Test Results

In silico test outputs can be generated by varying in silico test inputs. For example, an in silico simulation of a PFGE test accepts at least two inputs: a string sequence representing DNA, and a “digital restriction enzyme” that cuts the DNA at recognized patterns. The in silico PFGE test outputs a digital representation of a resulting PFGE banding pattern. Different output digital banding patterns can be produced by varying the sequence used as input into the in silico algorithm.

The following strategies can be employed to generate different input sequences to an in silico test:

Strategy 1

Observe a plurality of laboratory tests with known input DNA sequences and observe which input sequences produce one-away output results. Record each observed one-away event. If two known input DNA sequences produce a one-away laboratory test result, then the corresponding in silico test shall also produce a one-away in silico test result assuming that the two input string sequences accurately represent the DNA input into the laboratory test.

Apply each previously observed genetic event to all previously observed DNA sequences. For example, say that four DNA sequences have been previously observed—Sequence A, Sequence B, Sequence C and Sequence D—and that Sequence A and Sequence B are the only sequences that are one-away from another observed sequence. Note the single event that differ between Sequence A and Sequence B. Now, apply that same event, if possible, to each of the other remaining previously observed strings. For example, suppose that the single event difference between Sequence A and Sequence B is a single nucleotide polymorphism in a known gene in a known location. If Sequence C possesses that gene, then transform Sequence C by applying the same single event to Sequence C. The new resulting sequence, Sequence E, has not yet been observed in the laboratory. At this point, Sequence E is an artificial construct of our input sequence generation strategy. Now, perform the in silico test using Sequence C as input and then again using Sequence E as input. Since we know that Sequence C and Sequence E are one-away because we purposefully constructed them to be one-aways, we know that the in silico test outputs will represent two test results that are one event away. Next, we can compare Sequence E to Sequence B to see if those two sequences are one-away's. If they are one-away's, there is no need to rerun the in silico test results; in silico test results for Sequence A and Sequence E already exist. However, if we know that Sequence A and Sequence E are one-aways, then we can record that their resulting in silico test results represent two test outputs that vary by one genetic event.

Then, even though Sequence E has not been observed in an actual laboratory test, we can accept Sequence E as a potential input sequence. Therefore, when we observe a new actual one-away event we can still apply the one-away event to both a previously observed sequence (such as Sequence A) and also apply the one-away event to a potentially observed sequence (such as Sequence E.)

The purpose of this strategy is to generate a library of potential in silico one-away test results that can be used to compare against actual laboratory test results to rapidly determine if two actual laboratory results are separated by one genetic event.

Strategy 2—Brute Force

Given a string sequence, sequence A, a computer can generate all possible one away genetic events from the initial string input (x1, x2, x3, etc.) Accepting each input string, x1, x2, x3, etc, the computer could produce the in silico output for all sequences that are one event away from the initial string sequence. The in silico test results and the one-away relationship would be stored in a database.

It should be noted that neither the original input sequence nor the generated one-away string sequences must be an entire genome. Instead the input sequence may be represent a DNA sequence significantly shorter than that of an entire genome.

Strategy 3

An algorithm may use a priori knowledge to modify the string sequences input into the in silico experiment to generate new one-away sequences. Such a priori knowledge might take into account how DNA has been observed to have changed previously.

For example, the common molecular typing method known as spa-typing involves DNA sequencing a region of DNA from the S. aureus Protein A (“spa”) gene. It is known a priori that the sequenced region of the spa gene has a propensity to mutate and that the observed mutations included SNPs at specified locations and also the insertion or deletion of contiguous strings of DNA known as variable number of tandem repeats (“VNTRs”).

Given a string sequence representing the spa gene as input, the computer algorithm would intelligently generate new one-away DNA sequences using on a priori knowledge of how the spa gene naturally mutates.

Similar to the other strategies, these in silico generated one-away sequences can be used to generate in silico test results that will be known to be one-away from the other.

Strategy 4

Given two laboratory test results where the comparison of the two output results cannot definitively identify a one-away relationship, DNA sequencing can actually be performed on the original laboratory inputs to determined definitively whether the two laboratory input sequences are one-away's. One may choose to sequence an entire genome of a particular organism or sequence only a smaller subset of organism's genome.

Strategy 5

This strategy is similar to strategy 1 except that sequences other than one-away's are considered. Using algorithms that compute “edit distances”, two sequences can be compared to catalog all possible events that could have transformed one sequence into another. Each of the transformation events can be considered a single event, and each of those events can be applied individually, one at a time, to all previously observed sequences and all previously computer generated sequences similar to the process outlined in step 1.

### General Algorithm to Compare Laboratory Test Results

In a preferred embodiment, a general algorithm for comparing laboratory test results may comprise the following steps:
1) Conduct a laboratory test on DNA collected from organism 1.
2) Conduct same laboratory test on DNA collected from organism 2.
3) If the result of the first laboratory test is identical to the result of the second laboratory test, then record that the two organisms are identical and exit algorithm.
4) Compare each laboratory test result to a database of previous laboratory test results. If both laboratory test results are found in the database, then output whether the test results are “one event away” or “more than one event away” and exit algorithm.
5) Compare each laboratory test result to a database of all generated in silico test results. If both laboratory results match in silico test results in the database, then output whether the two in silico test results are “one event away” or “more than one event away” and exit algorithm.
6) Analyze the two laboratory test results to determine whether the two laboratory test results are one-away's. Use algorithms such as those listed in the section “Inferring Single Genetic Events from Laboratory Tests other than DNA Sequencing”. Prior knowledge of how the laboratory test expresses DNA may be sufficient to determine whether the two results are one event away from the other. If it can be determined that the results differ by one event or more than one event, then output the test result and exit the algorithm.
7) Perform actual DNA sequencing on the DNA that was used as input to the original laboratory tests. Examine the two DNA sequences and record whether the sequences differ by one genetic event or more. Then conduct the in silico experiment using the two DNA sequences.

Record all results and relationships in the database so that the analysis can be used as look-ups for future analysis.

### Focusing the Lens

It should be noted that current technology allows DNA microarray technology to query one SNP or tens of thousands of SNPs. Scaling the technology could allow microarrays to query millions the existence of millions of SNPs in a single test. The more SNPs that are queried in a microarray test, the less likely that two microarray test results shall differ by a single event. For example, suppose that a microarray is constructed to query input DNA for the existence of ten (10) sequences. If two distinct test results are identical in nine (9) array positions, and differ in one (1) array position, then those two results are one-away. Suppose that a different microarray test is constructed to query input DNA for the existence of ten thousand (10,000) sequences. If two distinct test results are identical in nine thousand nine hundred and 99 (9,999) array positions, and differ in only one (1) array position, then those two results are one-away. In general, it is more likely that the test that first example can find exactly nine (9) identities, than the second test finds exactly 9,999 identities.

When used to differentiate among closely related organisms, a laboratory test can be designed to look for sequences in such a manner that the test is not too “sensitive”. An overly sensitive test would identify more than one genetic between a plurality of input DNA. Whereas the opposite would be a laboratory test that produced too many identity results. A laboratory test can be designed for each facility to suitably differentiate among organisms be recognizing one-away events.

This process is analogous to the concept of course graining used in physics. The laboratory test can be designed to provide sufficient resolution without being too sensitive or not sensitive enough. Furthermore, all laboratory tests can be designed specifically for each organism and each facility. For example, a DNA sequencing test could be designed to query a single loci or possibly several carefully selected loci. However, it would be less likely that one-away events would be identified if DNA sequencing entire genomes. Specific loci and sequences would be sequenced for each organism. Another example would be to construct a PFGE laboratory test with one or more specifically selected restriction enzymes for each organism. Another example would be to create a microarray specific to each organism that queried a limited number of loci that were well selected knowing that they had a propensity to mutate.

Just as the design of a laboratory test and the region of DNA queried affects the frequency that genetic events are observed, the choice of a which laboratory test is selected will affect the ability to determine individual genetic events. As discussed previously, a PFGE test does not have the same output resolution as a DNA sequencing test. A laboratory test can be specifically designed, or “tuned,” to be most accurate in a given environment. This process is akin to focusing a lens to achieve optimum specificity for a given environment.

A laboratory test can comprise a plurality of laboratory tests performed in tandem. As an example, hospitals may have an endemic clone of pathogenic bacteria that infects a plurality of patients. A first hospital, Hospital A, may have an endemic clone of bacteria, Bacteria A, and a second hospital, Hospital B, may have a different endemic clone of pathogenic bacteria, Bacteria B, that is unrelated to the first Hospital A's endemic clone. One type of laboratory test may not detect any genetic variations among any of Hospital A's strains; they may all appear identical. However, that same laboratory test may observe many genetic events among Hospital B's endemic clone. A different laboratory test may detect genetic differences among Hospital A's endemic clone and not identify any genetic differences among Hospital B's endemic clone. A third laboratory test might be too sensitive so that all of Hospital A's endemic clone appear a being more than one event away.

Ultimately, in a preferred embodiment, enough single genetic events are identified among closely related strains such that the genetic events do not occur too frequently or too infrequently. Laboratory tests can be designed specifically for a given environment to meet this goal. The choice of which laboratory tests to use, and which regions of DNA to observe, can be customized, and focused, for a particular environment.

### Stochastic Processes/Markov Chains/Random Walks

The mutation of nucleotide molecules is a discrete-time stochastic process that can be modeled mathematically as a Markov chain or random walk. The arrangement of all possible DNA nucleotide molecules comprises the sample space, Ω. Each specific configuration of DNA nucleotide molecules is considered to be a system state. The transformation from one DNA sequence to a new DNA sequence describes a transition process that can be assigned a numerical probability. The sum of all probabilities of all possible transitions necessarily sum to 1, or 100% likelihood. Each state space transition can be assigned a probability and that probability recorded in a transition matrix. These characteristics define a “Markov Property”.

A first order Markov process states that only the last state occupied by a process is relevant in determining the future behavior of the process. Thus, the probability of transitioning to a new process state depends only on the state currently occupied. Equivalently, the future trajectory of a process depends only on the present state of the process. Such first-order Markov processes are described as being “memory-less”, because the process “forgets” about all previously occupied states after the process has transitioned to its current state. The future trajectory of the process only depends on the current state and not any historical state.

The one-away algorithm described herein reveals a first order Markov process wherein each DNA sequence represents a state space and each genetic event represents a transition to a new state space. Laboratory tests that express an organism's DNA describe a single state of a Markov process. The state may be expressed as a string sequence that represents nucleotide molecules observed at one or more loci, or the state may be expressed as an image-based banding pattern, or the state may be expressed as binary microarray results, or the state may be expressed by another analyzable output format that represents the original input DNA. Each laboratory test result represents a single process state at a particular instance in time.

The expression of all or some of an organism's DNA may be used to represent a state of an entire organism at a particular moment in time. The transition from one DNA state to another embodies the transition of an entire organism from one state to another, wherein the parent organism maintains the original state and the child offspring inherits the new, transitioned state.

In preferred embodiments, the systems and methods described herein interpret laboratory test results to observe, discover and interpret transitions between states. In order to discover a state transition, a laboratory test must be performed on at least two samples so that it can be determined whether one state may have transitioned into the other state. The methods and systems described herein determine whether i) two states are identical, ii) whether there may have been a direct, single transition from one state to the other state, or iii) whether there was more than one transition event from one state to the other. The methods and systems described herein can be used to discover single transitions between states without necessarily knowing the exact nature of, or composition of, each state.

The observation of transitions between process states can be recorded to form a transition matrix that represents the probability of one state transitioning to another. The result is a Markov transition matrix. A transition probability matrix of all possible transitions—observed or not—can be constructed by understanding, and estimating, how the laws of physics might influence the transition probabilities. It is understood that not every state, and therefore not every transition, is physically possible. Additionally, it may not be computationally feasible to consider every possible state transition; an infinite number of events exist when one considers the insertion of all possible DNA sequences at any point into the current state. A Markov chain can be formed by “chaining” together single state transitions where the future state is only dependent on the state immediately preceding the present state of the Markov process.

### Entity Relatedness

The methods and systems described herein distinguish among closely related states of any entity that may be described by a state that may change dynamically. Such entities may include but are not limited solely to organisms. Such state changes may be analyzed using common Markov chain techniques by first considering all possible single state transitions, and then considering all subsequent chained single state transitions.

A DNA sequence comprised of multiple nucleotide molecules may undergo a single genetic event thereby transforming into a second related DNA sequence. The original DNA sequence may be described as the “parent” and the resulting DNA sequence may be described as the “child”. Other terms that connote lineage such as ancestor or off-spring are also common. Each single DNA event can be assigned a probability, or likelihood, to occur. The occurrence of a single genetic event may be more or less probable than another different genetic event.

Two identical DNA sequences may each undergo a different and distinct single state transition, (a genetic event) that results in two distinct children DNA sequences. Two identical parents may produce two different and distinct children. One of these transitory genetic events might be a common, high-probability, event while the other transitory genetic event might be a rare, low-probability event. The high-probably event, or state transition, would be observed more frequently than the low-probability event as the high probability transition is more likely to occur when there are multiple entities that each occupy the identical initial state.

For example, albinism is the phenotypic expression of a low-probability genetic event. Given two identical parent DNA sequences, one parent sequence may experience a high-probability genetic event wherein the genetic event does not result in albinism. However, a second identical parent DNA sequence might experience a low-probability genetic event that does result in albinism.

The question can then be asked “which parent sequence is more closely related to the resultant child sequence?” Is the parent and the non-albino child more closely related than the parent and the albino child?

The answer is that both parent sequences are equally closely related to the respective resulting children sequences even though one of the observed genetic events is less probable than the other event. Each parent is one genetic event away from its child. Therefore we can define “relatedness” as the number of genetic events that separate two DNA sequences. If a parent begets two children, and one child has a rare mutation such as albinism, both children are still equally related to the parent. It is possible for a parent to transition to a child state, and then have the child state transition back to the parent state. In this scenario the parent state may actually be a descendant of another identical parent state.

The probability of a single genetic event represents the passage of time. A low-probability genetic event will occur and be observed less frequently than a high-probability genetic event. The probability of each genetic event can be approximated by observing a large number of genetic events. From such observations, it may be determined that certain genetic events are common, and some genetic events are rare. The transition probabilities are “approximated” because all genetic events must be considered possible, no matter how small the probability, and just not observed.

The methods and systems described herein seek DNA sequences separated by a single genetic event regardless of the laboratory test that expresses the single genetic event. Similarly, the algorithm described in this application, seeks DNA sequences separated by a single genetic event as opposed to other edit distance based algorithms which consider total edit distance, weighted edit distance and other metrics.

### Network Graphs

An undirected network graph can be created from the output of the “one-away” algorithms described herein. A graph is an abstract representation of a set of objects wherein some pairs of the objects are connected by links. An undirected graph connects objects, represented as vertices, with symmetric links represented by edges connecting the vertices. A symmetric link can be traversed in either direction whereas an asymmetric link may only be traversed in one direction. An asymmetric graph is also called a directed network graph.

A graph can be created from a Markov transition matrix wherein the vertices of the graph represent the individual process states and the links connecting the states represent the transitions between states. The vertices of an undirected network graph may represent the results of a laboratory experiment or the vertices of the graph may represent an actual organism on which the laboratory experiment was performed. The edges of an undirected network graph shall connect two vertices if the one-away algorithm determines that respective vertices are one event away from each other.

It is important to note that a current system state may have resulted from the transition from one of many prior states. For example, State A may have transitioned into State C and State B may have also transitioned directly into State C. In a set of observed laboratory data, it is desirable to learn whether the state prior to State C was State A or State B. A transition matrix calculated from previously observed data contains the probability that State A transitioned into State C and also the probability that State B transitioned into State C. Either transition may be theoretically possible, but only one of the transitions may have occurred in a given set of observed data.

To determine whether a state transition occurred from State A to State C or from State B to State C, additional laboratory experiments must be carried out. For example, State A, B and C may represent a component of the whole entity such as when States A, B and C are the different nucleotide compositions of a given gene's DNA. State A, B and C may have been collected from different strains of a common organism. Since State A, B and C, in this example, represent a component of the whole entity, then a second laboratory experiment can be conducted on a second component of the whole entity, such as a second gene, to determine if the states represented by the second laboratory experiment are shared by some but not all of the organisms. Observing whether certain strains share secondary state characteristics whereas other strains do not share the characteristics may provide hints to whether one state directly preceded another state.

For example, suppose a first laboratory experiment is conducted on three strains of an organism (Strain 1, Strain 2 and Strain 3) and that the experiment results show that Strain 1 is in State A, Strain 2 is in State B and Strain 3 is in State C. Furthermore, it is determined that State A is one event away from State C and also State B is one event away from State C. Then a second laboratory test can be performed on the same three organism strains. If two of the second laboratory test results are identical, and the third state is different, then it is more likely that a transition event occurred between the strains that share common results from the second laboratory experiment. For example, if Strain 1 and Strain 3 share an identical second test result, and that test result differs from the result of the experiment on Strain 2, then it can be assumed that the actual observed transition was between Strain 1 and Strain 3 and not Strain 2 and Strain 3.

### Creating a Directed Network Graph

If State A and State B are separated by a single state transition, then an undirected network graph is symmetric because the transition from State A to State B has the same probability as the transition from State B to State A.

However, given the discrete-time stochastic nature of these state transitions, we know that in actuality either State A preceded State B or that State B preceded State A. It can be difficult, but not impossible, to determine the temporal direction of a state transition.

If the temporal direction of a state transition can be determined, then a directed asymmetric network graph can be created. In an asymmetric network graph, the transition probability from State A to State B may not be the same transition probability from State B to State A.

Determining symmetric transition probabilities is easier than determining asymmetric transition probabilities, and thus creating undirected network graphs will be easier than creating directed network graphs. In the one-away algorithm described in this application, network graphs are built from states that are exactly one event away from the other state. The observation of a one-away event between two states does not imply a temporal element or describe which state preceded the other state.

An asymmetrical transition might be implied by observing which state occurred first in time, although the first observation of a state may not be sufficient evidence to determine that the first observed state did transition to the second state. Additional observations may lead to the conclusion that one state did transition to another second state. For instance, suppose a hospital patient in bed A experiences a bacterial infection characterized by State A on day 1. And suppose that same patient continues to experience the same bacterial infection on day 2 except that the state of the bacterial infection on day 2 is characterized as State B, and State B is one event away from State A, then the logical conclusion is that State A transitioned to State B, and the calculated transition probability can be assigned to the asymmetric transition from State A to State B.

Additionally, as noted above, each species and each region of DNA may have its own set of specific DNA event mutation rules that further specify the definition of a one-away event algorithm. For instance, one of the one-away events recognizes the insertion of any DNA sequence into a given sequence, and another rule recognizes the deletion of any DNA sequence from a given sequence. A more specific version of the insertion rule specific to a species or region of DNA might be that a contiguous region of DNA whose length is a multiple of 24 base pairs can be copied into the original sequence at a position adjacent to the original sequence being copied. Another rule might be any contiguous DNA sequence whose length is exactly 24 base pairs long can be deleted from the original sequence.

These two specific rules satisfy the definition of the one-away events. However, these two rules are asymmetric because the application of the specific insertion rule followed by the specific deletion rule will result in a different sequence if the deletion rule is applied before the insertion rule. For example, with asymmetric specific one-away rules, we can deduce that DNA sequence A must be the precursor of DNA sequence B because the specific rules do not allow for sequence B to change into sequence A. Such asymmetric rules may also assign directions between nodes on the network graph.

### Clock Speed

Two very closely related organisms may differ from the other by more than one genetic event even though one organism is a direct descendant of the other. Multiple single genetic events may occur between observation times. Microbial replication, for instance, occurs millions of times a second and, as part of the normal replication process, many genetic mutations may occur, albeit temporarily, as the mutations are either “corrected” or they do not survive.

As discussed above in the section “Focusing the Lens”, a laboratory test may be designed to only observe some but not all states of an organism. For example, the spa-typing laboratory test observes the state of a one particular region of DNA in Staphylococcus aureus. The spa-type test does not observe the state of other regions of DNA in the S. aureus genome, nor does it observe other states of the organism unrelated to the organism's DNA genome.

Different regions of an organism's genome may change or mutate at different rates. Therefore, one may observe more single genetic events in a given time frame in one region of an organism's genome, than in another region of the same organism's genome in the same exact time frame.

Therefore, the region of DNA observed by the laboratory test will have an effect on whether genetic events are observed or not.

A laboratory test may be designed to observe one or more regions of DNA. The design of the laboratory test and which region of DNA that the test has been design to query affects how many genetic events will be observed. A test designed to observe a region of DNA with an infrequent mutation rate will observer fewer genetic events over time than a test designed to observe a region of DNA with a frequent mutation rate.

### Building a Phylogenetic Tree One Step at a Time

Classical phylogenic-tree-creating algorithms, such as maximum parsimony, maximum likelihood, Unweighted Pair Group Method with Arithmetic Mean (“UPGMA”), neighbor joining and distance matrix methods and others described below have been traditionally employed to build evolutionary trees among distantly related organisms.

This algorithm differs from the traditional algorithms because it builds a phylogenetic tree one step at a time from observed data of extremely closely related organisms.

The one-step away algorithm described here-in shares elements of characteristics with several of the aforementioned classical algorithms. The one step away algorithm is both “distance based” and “character based”.

The one-step away algorithm described here-in does not work with distantly related inputs or with even semi-distant related inputs. The one-step away algorithm also requires significant observed input in order to build a phylogenetic tree.

Parsimony, or minimum evolution, methods build phylogenetic trees by discovering the minimum number of evolutionary events that would generate the tree. The one-away algorithm builds a phylogenetic tree by observed single steps

### Other Phylogenetic Algorithms Include:

UPGMA and WPGMA—“Distance based” clustering algorithms that build phylogenetic trees by joining the two “nearest” clusters and then joining the next two “nearest” clusters until all clusters have been compared. The algorithm is similar to the one-away algorithm in that states of the closest distances are compared and linked, but those states are not necessarily one-step away (and rarely are). Traditionally, these methods are used to build phylogenetic trees comparing distantly related species.

Levenshtein—The classic Levenshtein edit distance algorithm is similar to the one-away algorithm. However, the Levenshtein algorithm calculates edit distance between any two input sequences. However, unlike the Levenshtein algorithm, the one-away algorithm only builds phylogentic trees by single observed steps. The one-away algorithm is not able to produce a phylogenetic tree if transition events are not observed.

Maximum Parsimony—A “character based” algorithm that is similar to one-away algorithm in that the preferred phylogenetic tree is the tree that requires the least evolutionary change to explain observed data. The concept is similar to minimum spanning tree and dynamic programming methods that attempt to minimize the length of component paths through a network. However, the one-away algorithm is concerned with discovering the minimum unit paths between two connected points rather than discovering the optimal path that connects two points separated by more than one edge.

Minimum Spanning Tree—In principal, Minimum Spanning Tree (“MST”) algorithm is similar to the one-away algorithm in that the MST algorithm attempts to determine a tree with minimal edge lengths. However, unlike MST, the one-away algorithm only considers vertices separated by one edge (one away). To the one-away algorithm, only the state that immediately precedes another state is important. The entire minimal path through a network is of lesser importance.

Maximum Likelihood—A “character based” algorithm that evaluates the probability that a proposed model (phylogenetic tree) matches observed data.

Neighbor Joining—Another “distanced based” iterative algorithm based on minimum evolution similar to UPGMA algorithm. However, whereas UPGMA assumes a constant rate of evolution, neighbor joining allows for varying evolutionary rates.

eBurst—A clustering algorithm created to analyze the evolution of bacterial clones. Developed to be used on MLST sequence data. The algorithm is better suited for global epidemiology than very closely related strains found in local epidemiology. The eBurst algorithm describes single locus variants which are similar to one-aways. However the single locus variants described in the eBurst algorithm may actually be separated by multiple genetic events as opposed to single one-away events.

### Using Other Tree Building Algorithms to Fine Tune

The one-away algorithm requires a plurality of observed data points to build a phylogenetic tree. Since vertices on the tree represent single events, it is possible that not all observed data is interconnected. Vertices that do not connect may represent truly separate evolutionary clads among closely related organisms. Or, vertices that do not connect may indicate that intermediary states were not yet observed. Classical phylogeny algorithms can also be employed to help determine relationships among clusters of data that do not connect via the one-away algorithm.

### Example

### Infection Control and Short Term Disease Investigations

### Traditional Disease Outbreak Studies

Healthcare practitioners conduct epidemiological studies in healthcare environments to discover disease outbreaks and clusters of related disease. Once identified, such clusters may elucidate sources of disease which can then be eradicated to prevent future disease spread.

Organizations such as the US Centers for Disease Control (“CDC”) and the World Health Organization (“WHO”) have established guidelines for conducting disease investigations that include protocols for data collection and statistical analysis. Well-established epidemiological practices require the collection of a statistically relevant amount of data so that accurate conclusions can be drawn.

Epidemiologists recognize that understanding pathogen distribution and relatedness is essential for determining the epidemiology of nosocomial infections and aiding in the design of rational pathogen control methods.

Traditional epidemiological studies result in a posteriori decision making because efforts to control further disease spread require the collection of sufficient data. In order to identify clusters of disease, a sufficient number of patients must become infected with disease before accurate conclusions based on statistical arguments can be made. Such a posteriori analysis may help identify and correct problematic sources of disease, such controlling an existing outbreak of disease. But, such a posteriori epidemiological studies do not prevent the disease outbreak from occurring in the first place.

Disease investigation training materials displayed on the CDC web site teach methods of collecting information after the disease outbreak has occurred. Such methods include interrogation techniques, analysis of the intersection of patient locations prior to infection and searching for signs of unreported infection. This traditional approach to disease investigation looks for clusters of disease after the disease has occurred.

New Paradigm for Controlling Infections

The methods and systems described herein are a novel system and method of controlling the spread of disease by directing infection control actions before statistically relevant clusters of disease are recognized. The methods and systems described herein predict disease spread. The methods and systems described herein employ the molecular profiling of pathogenic microbes to discover mechanisms of pathogen transfer so that infection control actions can be directed towards eliminating transfer mechanisms and also eliminating pathogen sources.

Identifying and eradicating pathogen sources will remain an important component of infection and disease control. However, in healthcare environments, the pathogen source is often previously infected patients. Since, except under extreme and costly measures, patients cannot be removed from healthcare environments, the methods and systems described herein focus on preventing the transfer of pathogens rather than focus on the complete elimination of the pathogen from the environment.

Modern healthcare facilities such as hospitals and long term care facilities may be understaffed and lack sufficient clinical resources to focus significant time and money on infection control. Infection control practitioners typically react to infections after the fact rather than trying to prevent future infections. Additionally, standard infection control practice does not direct infection control actions based on detailed knowledge of the infecting organism. The methods and systems described herein apply a method of determining relatedness among closely related entities in order to disrupt the flow of pathogens in a healthcare environment. Other applications of the methods and systems described herein also apply when entities can be described by discrete states and dynamic transitions between states exist.

### Sources and Vectors

Included within the organisms whose source and/or transmission may be studied according to the invention are pathogens. A pathogen source, the reservoir which harbors infectious agents, may be a living organism or an inanimate object. A living organism may be infected by the pathogen or the living organism may carry the pathogen without having been infected by the pathogen. A person who hosts the pathogen but who does not have an infection is called a “carrier.” An uninfected person who hosts a pathogen is referred to as being “colonized” by the pathogen. A person or inanimate object on which the pathogen temporarily resides is considered to be “contaminated.” A person may be contaminated without being a carrier or being infected.

A pathogen vector is the mechanism by which a pathogen is transferred from an originating source to a susceptible host. A vector may transfer a pathogen from an originating source to an intermediary source before infecting a susceptible host. The intermediate source may be a living organism or an inanimate object. The intermediate source may also become a carrier or may become infected, although the intermediate source may become infected after the susceptible host becomes infected.

The methods and systems described herein act to identify the source that immediately precedes an infected organism, and to identify the transfer mechanism by which the pathogen moved from the infecting source to the susceptible host. The methods and systems described herein primarily act to direct actions that shall eliminate the mechanism of transfer as well as possibly eliminating the originating pathogen source.

Vectors may act as both transfer mechanisms and sources simultaneously. For instance, in a healthcare environment, a nurse who is colonized by a pathogen but not infected can act as a pathogen source and can also transfer that pathogen to another susceptible person.

A posteriori analysis of data may identify clusters of infection by recognizing a common disease source. Once identified, the source may be eliminated thereby eliminating the spread of future disease from that source. For instance, in a healthcare environment, it may be noted that a number of patients undergoing dialysis may all share a common infection leading one to believe that a dialysis machine is the source of the infecting pathogen.

In a healthcare environment, such as a hospital, infected patients will always be a pathogen source but patients cannot be eliminated from a hospital. Therefore, the most obvious pathogen source, the patient, will always exist in a healthcare environment. Infection control strategies exist to segregate and isolate patients from the general hospital population, but in reality, the pathogen source still exists.

In preferred embodiments the methods and systems described herein provide for identifying and eliminating the mechanism by which pathogens move. Since infected patients are a primary pathogen source, and since we can never eliminate patients, we shall focus on discovering and eliminating the means by which pathogens move from a source to an uninfected host.

### Pathogen Sources

A pathogen source may be indigenous or foreign to a particular healthcare environment. Possible pathogen sources are:
1) The patient may “self-infect” if the patient is a pathogen carrier
2) Another patient. The patient may be infected or colonized
3) A clinical worker in the healthcare environment such as a doctor or a nurse. The clinical worker may be infected, colonized or contaminated
4) A non-clinical worker in the healthcare environment such as a dietician or a janitor. The non-clinical worker may be infected, colonized or contaminated
5) A civilian, such as a visitor, in the healthcare environment. The civilian may be infected, colonized or contaminated
6) A contaminated inanimate object, either indigenous or foreign

### Vectors

Vectors are the mechanism by which a pathogen is transferred from one source to another. Different pathogens spread by different modes of transmission including direct contact, ingestion, or respiratory.

### Historical Baseline—Molecular Fingerprinting

As previously discussed, certain laboratory tests may output an organism's genotype or a phenotype.

To understand which pathogens and pathogens exist, and have existed, in a facility, investigators should determine the genotypes and phenotypes of as many pathogens that exist in the facility and store this information in a computer database. Additionally, clinical healthcare data such as patient demographic data, patient clinical data, patient movement, pathogen-related data, clinical and non-clinical healthcare worker data including hours worked should also be collected and stored in a computer database. This database shall serve as a historical snapshot of what has already happened at the healthcare or other facility.

Identify Likelihood of Infection

Different patients, upon admission to a healthcare facility, have different likelihoods of obtaining an infection from a pathogen. Each patient can be assigned a dynamic numerical value that relates to the relative likelihood that he or she will obtain an infection. Standard statistical mathematics techniques for analyzing and comparing likelihood ratios apply.

The relative likelihood that a patient may obtain an infection while at the hospital may be based on many risk factors. Each risk factor may be assigned a numerical weight. The sum of each weighted risk factors can be compared to another patient to determine the relative likelihood that one patient will obtain an infection compared to another patient. Physical observations of when patients with a given set of risk factors acquire an infection can lead to the calculation of the likelihood φ.

Certain risk factors only affect a particular individual such as comorbidities and age. For example, a person's age is a risk factor to that person only. Certain risk factors may be shared among several patients. For example, shared risk factors may include beds shared by different occupants at different times, shared inanimate objects used in treatment, shared facilities, shared clinical and non-clinical workers providing treatment related services, and also proximity to other infected, colonized and contaminated living beings and inanimate objects.

The likelihood that a patient will obtain an infection is a dynamic, continuously changing value. As a patient becomes healthier or sicker, the patient's likelihood of infection will change. Similarly, as shared risk factors change, such as the contamination of a shared inanimate object used in treatment during the course of a patient admission, the likelihood that a patient or patients will obtain an infection also changes. The likelihood that a patient will obtain an infection is a stochastic event that can be monitored in much the same manner that an individual stock on a stock market can be monitored.

Individual risk factors also affect the likelihood that an individual patient will obtain an infection. Individual risk factors do not affect whether any other person obtains an infection other than that one individual. Of course, once an individual acquires an infection or becomes colonized or contaminated with a pathogen, he/she becomes a shared risk factor to other patients. Individual risk factors have been well identified in the medical literature, and numerous epidemiology studies have been conducted to observe these individual risk factors.

Shared risk factors are created from the presence of pathogens. A sterile environment with no pathogens has no shared risk factors. Therefore, in an environment absent of pathogens, shared risk factors do not contribute to the likelihood that a patient will obtain an infection. In an environment completely absent of pathogens, there is a zero probability of a patient obtaining a microbial infection from the environment. The only possibility of infection in an otherwise sterile environment is an individual risk factor—if the patient is colonized. Of course, over the course of time pathogens may be introduced to the environment thus adding shared risk factors. Therefore,

Total Risk Factor Score=

ΣWeighted Shared Risk Factorⱼ+ΣWeighted Individual Risk Factor

Shared risk factors require the presence of pathogens. A patient may have several risk factor scores that are specific to 1) possible infecting pathogens and also 2) possible strain of infecting pathogens. Furthermore, both individual risk factor scores and shared risk factors scores may be specific to each pathogen or each strain of each pathogen.

For example, some patients may be more likely to be infected by a particular pathogen strain. Suppose several patients in hospital ward X have acquired an infection caused by strain A of S. aureus and suppose several patients in hospital ward Y have acquired an infection caused by strain B of S. aureus. A new patient is admitted to hospital ward X. Then that new patient shall be more likely to acquire an infection from Strain A than from Strain B. Thus, in this example, two separate risk factor scores shall be tallied—the likelihood of acquiring an infection from Strain A and the likelihood of acquiring an infection from Strain B. Furthermore, certain risk factors may be weighted differently depending on the possible infecting pathogen. Therefore, this method can create a relative score that identifies the relative likelihood that a patient shall be infected, or colonized, in the future by a particular pathogen or a particular pathogen strain.

Individual risk factors contribute to the likelihood that a patient obtains any infection, and shared risk factors contribute to the probability that a patient obtains an infection from a specific pathogen strain. Individual risk factors may be weighted differently depending upon the possible infecting pathogen.

Different environments shall have different shared risk factors. Hospital A may have different shared risk factors than Hospital B. Also, shared risk factors change over time. In a single facility, infected patients change locations, patients are discharged, new patients with prior infections will be admitted to the healthcare environment, colonized civilians will visit the hospital, healthcare workers will randomly become colonized and decolonized, and so on. Shared risk factors shall be dynamic. It may be impractical to measure and record all conceivable data points at all points in time.

Although individual risk factors may change during the course of a patient's admission to a healthcare facility, individual risk factors can be easily monitored. Because it may not be practical to monitor and record every conceivable shared risk factor, shared risk factors may be implied by comparing the observed individual risk factors from infected and freshly colonized patients or clinicians. Clinical metrics, such as primary diagnosis, existing co-morbidities and prior conditions of infected patients can be compared and laboratory tests results that identify infecting pathogen genotype and phenotype can be compared. From these comparisons, inferences can be made about potential common shared risk factors.

For example, two patients who share common or similar diagnoses are more likely to be treated by the same clinicians, share common treatment regimes, occupy similar locations and encounter common visitors because the visitors are visiting shared locations. An assumption can be made that a common originating pathogen source may have infected two or more patients when those patients share a similar diagnosis and when the infecting pathogens have an identical or very closely related genotype or phenotype. When infected patients share a similar diagnosis, and when infecting pathogens are very closely related, then specific shared risk factors should be identified. Once identified scores associated with those common risk scores can automatically be applied to other patients who share the common risk factors.

If common shared risk factors are identified, then the relative likelihood that another uninfected patient with some or all of the same shared risk factors shall be infected by the similar pathogen strain shall increase. The algorithm that determines the relative likelihood that an uninfected patient shall acquire an infection from a specific pathogen strain should assign a greater weight to the risk factors of patient's who most recently acquired an infection. Also, this algorithm should assign a greater weight to those shared risk factors that are closer in physical space to the uninfected patient. By giving greater weight to those shared risk factors which are closer in space and closer in time, the algorithm self-adjusts. For example, the contribution to the calculation of a shared risk factor score shall be greater from an infected patient in close proximity to an uninfected patient than contribution from an infected patient a greater distance away. Similarly, the contribution to the calculation of a shared risk factor score shall be greater from a patient with a recent infection than from a patient who acquired an in infection in the past. Again, risk factor scores can be calculated for every possible pathogen and also every strain of every pathogen.

### Endemic Strains

This predictive algorithm can consider which existing infections are closest in space and closest in time to uninfected patients. In a healthcare facility, such as a hospital, there may be a preponderance of certain indigenous pathogen strains. Such endemic strains typically outnumber all other strains of a particular pathogen species.

When endemic strains exist at a facility, the algorithm may correctly predict that the most likely future infection shall result from an endemic strain. Therefore, when endemic strains exist in a facility, investigators should employ laboratory tests that observe hyper-variable genotypic and phenotypic characteristics of a pathogen to discriminate among endemic strains. This results from the discussion presented earlier in the section “focusing the lens.”

Because endemic strains may exist in many locations in a healthcare facility, the algorithm shall give more weight to pathogen strains that are closest to each uninfected patient in both space and time.

When endemic strains exist at a facility, it may be difficult to determine an originating pathogen source if newly observed infections were acquired from an endemic strain. However, if the newly infecting pathogen is not an endemic strain, then it may be very easy to identify an originating source. Endemic strains shall produce similar genotypic and phenotypic laboratory test outputs. When an infecting strain is not endemic, a laboratory test performed on a non-endemic strain shall produce a test output that looks different from the results of laboratory tests performed on endemic strain. Therefore, against the consistent background of endemic strain laboratory test results, it is very easy to identify, if they have been previously observed, other pathogen strains that are very closely related to the newly infecting pathogen.

For example, suppose that the endemic strain in a facility is characterized as “fingerprint A”, and 75% of strains collected at the facility have the “fingerprint A” genotype. When a strain with “fingerprint B” is observed at the facility, then this strain can be easily identified when compared to the endemic strain “fingerprint A” laboratory test results. When a second strain with “fingerprint B” is collected at the facility in a time frame close to when the original “fingerprint B” strain was observed, then is more likely that the first observed “fingerprint B” strain with was the source of the second “fingerprint B” infection.

A reasonable question would be “from where did the first strain with Fingerprint B” come if it had not been seen before at the facility?” The answer is: the first strain collected of a particular fingerprint may have been introduced to the environment from a source external to the hospital such as: the patient herself, if she was colonized upon entering the hospital; a colonized healthcare worker; or, a colonized civilian who introduced the pathogen into the healthcare environment from the outside community.

If it is determined that a newly infecting pathogen has a similar genotype or phenotypes to an endemic strain, or if the newly infecting pathogen has the properties that are common to many other strains at the facility, then it will be necessary to perform a different laboratory test with better resolution that can discriminate among the otherwise identical strains. Similar to the previous discussion in the section “Focusing the Lens”, a different laboratory test with greater specificity may be able to differentiate among otherwise seemingly identical strains.

### Network Graphs

Disease transmission can be represented visually by generating a directed network graph. Graph nodes represent pathogen sources and the connecting directed graph edges represent transmission events.

Determining Transmission and Preventative Intervention—An uninfected patient may acquire an infection from the following generic sources:
1) The patient himself/herself
2) Another patient
3) A clinical worker in the healthcare environment
4) A non-clinical worker in the healthcare environment
5) A civilian, such as a visitor, in the healthcare environment
6) An inanimate object, either indigenous or foreign

Of these above sources, people may be infected, colonized or contaminated. Inanimate objects may be contaminated. Transmission occurs when a pathogen moves from a source to a target via a vector. Transmission may result in a new infection, a new colonization, a new contamination or a non-event. Transmission events may be recognized by generating a directed network graph where nodes represent sources and edges represent transmission events. Potential transmission events may be recognized by identifying pathogen sources with identical genotypes or phenotypes, or very closely related genotypes or phenotypes as has been discussed earlier.

Identifying identical or very closely related genotypes or phenotypes may not absolutely identify originating sources or vectors. However, other clinical data may be observed to further refine the selection of a possible source and a possible transmission vector.

Each possible source should be assigned a numerical score whereby a greater weight is assigned to those possible sources that share a closer proximity in time, a closer proximity in space and also share similar elements of clinical data. Each possible vector from each possible source to the newly infected or colonized patient should be assigned a score based on observations of similar risk factors. A possible source must be infected, colonized or contaminated with an identical or very closely related organism as the newly infected person.

By assigning scores to possible sources, and to possible vectors, the algorithm can suggest which possible source is most likely and which vector is most likely.

Based on the suggested methods of transmission, healthcare personnel can take specific actions to eradicate or sterilize the means of transmission based on analysis of both shared and individual risk factors. For example, the algorithm may suggest that patients with a certain diagnosis or treatment method are more likely to self-infect if they are previously colonized. Then, for other uninfected patients who are previously colonized and who share a common diagnosis or treatment method, healthcare practitioners should take extra actions to ensure that self-transmission is prevented. Such methods might include established techniques such as Chlorhexidine bathing, Antimicrobial-impregnated catheters, and Chlorhexidine-impregnated dressings and proper sterilization of skin and inanimate treatment equipment.

After a previously uninfected patient has acquired an infection, it may not be possible to absolutely determine the actual pathogen source and the actual transmission vector. To the newly infected patient, it does not matter as he/she has already acquired a new infection. However by assigning a quantitative likelihood measures to every potential source and every potential vector, healthcare practitioners can direct their actions to eliminate future transmission events. Furthermore, a quantitative likelihood measure can be assigned to each uninfected person that indicates the relative likelihood that the uninfected patient shall acquire an infection while in hospital. This quantitative measure further directs infection control actions as healthcare practitioners can focus their strongest efforts on eradicating vectors that might infect those people who are most likely to acquire a new infection. The algorithm not only assigns a value to the relative likelihood that an uninfected patient shall acquire an infection from a particular pathogen, the algorithm also assigns a value to the relative likelihood that an uninfected patient shall be infected by a particular strain of a particular pathogen. Since different vectors may transfer different pathogen strains, a healthcare practitioner may focus specific sterilization actions based on factors including the following:
1) the greater likelihood that a patient shall acquire an infection from a specific pathogen strain;
2) the greater likelihood of a specific pathogen source;
3) the greater likelihood of a specific pathogen vector.

As a dynamic stochastic system, the computer algorithm can monitor a constantly changing set of input variables. The computer algorithm can produce discrete sets of values representing different likelihood measures to predict which events actually occurred and which events might occur in the future so that intervention actions can prevent those future events from occurring.

### Infection Control Analysis Decision System

In exemplary embodiments, an Infection Control Analysis Decision System (“ICADS”) can direct infection control actions to prevent and limit future pathogen transmission. In such a system, Bayesian statistical techniques can be applied to predict which actions will be most effective.

Patient healthcare metrics such as “apache II” score, assign a numerical value to patient disease severity. Many other patient clinical measurements can be assigned a “risk factor” value. Individual measurements can be assigned different weights and used to calculate an over-all patient risk factor value. Scores such as Apache II and patient risk factor score can represent the likelihood that a patient will obtain a future infection while in the healthcare facility. Essentially the sicker the patient, and the higher the patient risk, the more likely that the patient acquires a new infection.

The calculation of such likelihood scores are important. However, such calculation can be difficult to accomplish because the scores require the collection of many data points. Additionally, such likelihood scores only indicates the chance that a patient acquires any new infection as opposed to a specific infection. Therefore there is limited value in this score to direct infection control actions.

However, this current system calculates the likelihood that a patient will acquire an infection from a pathogen with a specific molecular fingerprint. For example, what is the likelihood that Patient X will acquire a S. aureus infection that has genetic fingerprint categorized as “1234”?

To do this, the system analyzes:
1) How likely the patient is to acquire any infection by considering patient risk factors, disease severity, etc as described above
2) The spatial-temporal density of each pathogen sub-speciated by the molecular fingerprinting techniques described in this invention

For example, a patient who has already acquired a S. aureus infection with molecular fingerprint categorized as “1234” would be recorded as having 100% of S. aureus infection with fingerprint “1234”. A patient who has not yet acquired an infection but who is near-by in space and/or time, such as a patient in an adjacent bed, or a patient in the same ward, might be assigned a score of 65% of S. aureus infection with fingerprint “1234”. Such likelihood scores would be assigned to every patient for every pathogen and for every molecular fingerprinting sub-species. A patient who has a 65% chance of infection from pathogen sub-species X has not yet acquired an infection. The same patient may have a 35% chance of acquiring an infection from a different pathogen of subspecies Y. Since the likelihood of acquiring an infection from specific pathogen subspecies X is greater than acquiring an infection from specific pathogen subspecies Y, the infection control analysis detection system will output specific actions that will better prevent the transmission of pathogen sub-species x to the particular patient. Each patient will have his or her own specific list of preventative infection control actions to best control the most likely future infections.

The decision control system is programmed to run on a computer system. The computer software uses Bayesian statistical techniques where calculation of the output likelihoods changes as new information is acquired and input into the decision making algorithm. Other than space-time coordinates of infected patient locations, and molecular fingerprint data from infecting specimens, there is no other additional data that must be used as input into the decision making algorithm. Of course, other clinical data can be input into the algorithm and used to improve the algorithm effectiveness.

What is claimed is:

1. A method of determining a source of, and/or tracking the transmission of, a pathogenic organism, the method comprising:
receiving, in a processing device, laboratory test results representing partial or complete nucleotide sequence or expression state data for a pathogenic organism in a first biological sample and in a second biological sample,
comparing, by a processing device, a genetic state data for the organism in the first biological sample to a genetic state data for the organism in the second biological sample;
determining, by the processing device, whether the first and second nucleotide sequence or expression states have a one-away relationship based on the partial or complete nucleotide sequence or expression state data for the pathogenic organism in the first biological sample and in the second biological sample;
recording, in memory in communication with the processing device, the relationship between the organism in the first and second biological samples if the first and second nucleotide sequences or expressions are the same or one-away; and
constructing, by the processing device, a representation of the transmission of the pathogenic organism based on connections between samples containing organisms having a one-away relationship.

2. The method of claim 1, wherein determining whether the first and second nucleotide sequence or expression states have a one-away relationship comprises determining whether the partial or complete nucleotide sequence or expression state data for the pathogenic organism in the first biological sample and in the second biological sample is the same and if not the same determining if the relationship is one-away or more than one-away.

3. The method of claim 1, wherein determining whether the first and second nucleotide sequence or expression states have a one-away relationship comprises comparing the first and second partial or complete nucleotide sequence or expression state data to records recalled from a database in memory in communication with the processing device of partial or complete nucleotide sequence or expression state data stored in a memory of the processing device, wherein the database comprises records of previously observed one-away relationships and/or in silico generated possible partial or complete nucleotide sequence or expression state data known to have a one-away relationship.

4. The method of claim 2, wherein determining whether the first and second nucleotide sequence or expression states have a one-away relationship further comprises comparing the first and second partial or complete nucleotide sequence or expression state data to records recalled from a database in memory in communication with the processing device of partial or complete nucleotide sequence or expression state data stored in a memory of the processing device, wherein the database comprises records of previously observed one-away relationships and/or in silico generated possible partial or complete nucleotide sequence or expression state data known to have a one-away relationship.

5. The method of claim 1, wherein constructing a representation of the transmission of the pathogenic organism comprises receiving in the processing device from a database in memory in communication with the processing device records comprising time and place data for the collection of the first biological sample and the second biological sample and connecting the first biological sample and the second biological sample only if the collection of the first and second samples occurred in a proximate time and place.

6. The method of claim 4, wherein constructing a representation of the transmission of the pathogenic organism comprises receiving in the processing device from a database in memory in communication with the processing device records comprising time and place data for the collection of the first biological sample and the second biological sample and connecting the first biological sample and the second biological sample only if the collection of the first and second samples occurred in a proximate time and place.

7. The method of claim 1, wherein constructing a representation of the transmission of the pathogenic organism comprises constructing a network graph or phylogenetic tree and outputting said network graph or phylogenetic tree to a display device interfaced to the processing device.

8. The method of claim 6, wherein constructing a representation of the transmission of the pathogenic organism comprises constructing a network graph or phylogenetic tree and outputting said network graph or phylogenetic tree to a display device interfaced to the processing device.

9. The method of claim 1, wherein receiving, in a processing device, laboratory test results representing partial or complete nucleotide sequence or expression state data for a pathogenic organism in a first biological sample and in a second biological sample comprises receiving said data by a receiving device, or receiving data for one or both of said first biological sample and in a second biological sample from a database in memory or a storage device in communication with said processing device.

10. The method of claim 1, further comprising identifying one or more sources of the pathogen and sterilizing or quarantining said source or sources.

11. The method of claim 1, further comprising identifying one or more pathogen transmission vectors and sterilizing or quarantining or removing or eliminating said transmission vector.

12. The method of claim 1, wherein conducting laboratory tests to determine partial or complete nucleotide sequence or expression state data comprises DNA sequencing, a pulse field gel electrophoresis (“PFGE”) laboratory test, a DNA microarray laboratory test, repPCR, MLVA, or MLST.

13. The method of claim 1 wherein comparing the partial or complete nucleotide sequence or expression state data comprises identifying a genetic event that is one of
a single nucleotide polymorphism, wherein a single nucleotide mutates into another nucleotide;
a single nucleotide deletion, wherein a single nucleotide is deleted from string sequence;
a single nucleotide insertion, wherein a single nucleotide is inserted into a string sequence;
a contiguous nucleotide sequence deletion, wherein one or more contiguous nucleotide sequences, comprising a single unit, are deleted from a DNA sequence;
a contiguous nucleotide sequence insertion, wherein one or more contiguous nucleotide sequences, comprising a single unit, are inserted into a DNA sequence;
a contiguous nucleotide sequence movement, wherein one or more contiguous nucleotide sequences, comprising a single unit, are moved from the original position to a new position in the same DNA sequence; and
a contiguous nucleotide sequence reversal, wherein several contiguous nucleotide sequences, comprising a single unit, are reversed at the original position or new position in the same DNA sequence.

14. A processor-readable medium having processor-executable instructions for performing a method comprising:
e) receiving a laboratory test result on DNA collected from a pathogenic organism in a first sample;
f) receiving a laboratory test result on DNA collected from from a pathogenic organism in a second sample;
g) if the result of the first laboratory test is identical to the result of the second laboratory test, then record that the two organisms are identical and stop;
h) if the result of the first laboratory test is not identical to the result of the second laboratory test, then analyze the two laboratory test results to determine whether the two laboratory test results are one-away by a method chosen from among
i. comparing each laboratory test result to a database of previously analyzed laboratory test results, and if both laboratory test results are found in the database, then look up and output whether the test results are one event away or more than one event away and stop,
ii. comparing each laboratory test result to a database of generated in silico test results, and if both laboratory results match in silico test results in the database, then look up and output whether the two in silico test results are “one event away” or “more than one event away” and stop, and
iii. analyzing the laboratory test results to determine whether the two laboratory test results are one-away, then output the analysis result and stop.

15. A system for tracking the path of an infection comprising:
a memory for storing first and second nucleotide sequences or expressions of nucleotide sequences determined from a pathogenic organism present in a first and second biological sample;
a processor configured to:
access the first and second nucleotide sequences or expression from the memory;
compare the first and second nucleotide sequences or expressions;
determine whether the first and second nucleotide sequences or expressions are the same, one-away, or not one-away;
connect the first and second biological samples if the first and second nucleotide sequences or expressions are the same or one-away; and
return a report of connected biological samples.

16. The system of claim 15, further comprising:
a database containing a library of nucleotide sequences or expressions,
wherein the processor is configured to compare the first and second nucleotide sequences or expressions to the database.

17. The system of claim 16, wherein the processor is configured to populate the database with in silico generated nucleotide sequences or expressions and to analyze the in silico generated nucleotide sequences or expressions to determine if the in silico generated nucleotide sequences or expressions are one-away.

18. An electronic system configured to carry out the method of claim 1 for determining a source of, and/or tracking the transmission of a pathogen, the system comprising:
a receiving device configured to receive a first laboratory test result on DNA collected from a pathogenic organism in a first sample and a second laboratory test result on DNA collected from a pathogenic organism in a second sample;
a processing device configured to
compare a genetic state data for the organism in the first biological sample to a genetic state data for the organism in the second biological sample,
store that the two organisms are identical if the result of the first laboratory test is identical to the result of the second laboratory test, or
analyze the two laboratory test results to determine whether the first and the second laboratory test results are one-away if the result of the first laboratory test is not identical to the result of the second laboratory test,
wherein the processor makes the determination whether the first and the second laboratory test results are one-away by one of
comparing each laboratory test result to a database storing previously analyzed laboratory test results, and outputting whether the test results are one event away or more than one event away if both laboratory test results are found in the database,
comparing each laboratory test result to a database of generated in silico test results, and outputting whether the two in silico test results are “one event away” or “more than one event away” if both laboratory results match in silico test results in the database, or
analyzing the laboratory test results to determine whether the two laboratory test results are one-away, and outputting the analysis result.

19. A method for determining regions of DNA suitable for one-way analysis, the method comprising:
receiving, by a receiving device, a plurality of pathogens;
performing, by a processor, genome sequencing of the plurality of pathogens;
comparing, by the processor, genome sequence of each of the plurality of pathogens with the genome sequences of all of the other plurality of pathogens of a same species;
identifying, by the processor, a DNA sequence for a gene coding region, the gene coding region being present in each of the genome sequences of the same species;
storing, in a database, the DNA sequence for every gene present in every genome sequences of the same species;
identifying, by the processor, all gene coding regions substantially present in each of the genome sequences of the same species;
storing, in a database, the DNA sequence for every gene substantially present in every genome sequences of the same species;
identifying, by the processor, all regions of DNA of the same species having a variable number of tandem repeats;
storing, in a database, the DNA sequence for every region having the variable number of tandem repeats;
identifying, by the processor, all single nucleotide polymorphisms in a conserved region among the genome sequences for the same species;
storing, in a database, the DNA sequence for every identified single nucleotide polymorphisms and the surrounding conserved DNA;
comparing, by the processor, similar regions of DNA;
determining, by the processor, a number of identical sequences from comparable regions of DNA and a number of variations among the comparable regions of DNA; and
selecting, by the processor, a plurality of regions to identify “one-away” events based on the number of identical sequences from comparable regions of DNA and the number of variations among the comparable regions of DNA.

20. The method according to claim 1, wherein the conducting laboratory tests to determine partial or complete nucleotide sequence or expression state data comprises DNA sequencing, the processing device determines whether the first and second nucleotide sequence or expression states have a relationship as same, one-away, or not one-away by
comparing the DNA sequence of the first biological sample to the DNA sequence of the second biological sample, and outputting that the two DNA sequences are identical when the two DNA sequences are identical,
searching a database storing relationships between DNA sequences, and outputting the stored relationship when the relationship between the two DNA sequences has been previously recorded as being one-away or more than one-away from the other,
checking to see if the DNA sequence of the first biological sample is a prefix of DNA sequence of the second biological sample, and storing the relationship as one-away in the database, and outputting one away when the first biological sample is a prefix of DNA sequence of the second biological sample,
checking to see if the DNA sequence of the second biological sample is a prefix of DNA sequence of the first biological sample, and storing the relationship as one-away in the database, and outputting one away when the second biological sample is a prefix of DNA sequence of the first biological sample,
checking to see if the DNA sequence of the first biological sample is a suffix of DNA sequence of the second biological sample, and storing the relationship as one-away in the database, and outputting one away when the first biological sample is a suffix of DNA sequence of the second biological sample,
checking to see if the DNA sequence of the second biological sample is a suffix of DNA sequence of the first biological sample, and storing the relationship as one-away in the database, and outputting one away when the second biological sample is a suffix of DNA sequence of the first biological sample,
checking to see if the DNA sequence of the first biological sample and the DNA sequence of the second biological sample are the same length, wherein
when the DNA sequence of the first biological sample and the DNA sequence of the second biological sample are the same length, comparing the two sequences to determine if the two sequences differ by a plurality of units, storing the relationship as more than one-away in the database and outputting more than one away when the two sequences differ by a plurality of units,
when the DNA sequence of the first biological sample and the DNA sequence of the second biological sample are the same length, and the two sequences differ by one unit, storing the relationship as one-away in the database, and outputting one away,
when the DNA sequence of the first biological sample and the DNA sequence of the second biological sample have different lengths, and when the two sequences share a common prefix and when the two sequences share a common suffix and when a concatenation of the common prefix and the common suffix exactly equals either the DNA sequence of the first biological sample or the DNA sequence of the second biological sample, storing the relationship in the database and outputting one genetic event away.

21. The method according to claim 1, wherein the conducting laboratory tests to determine partial or complete nucleotide sequence or expression state data comprises a DNA microarray laboratory test, the processing device determines whether the first and second nucleotide sequence or expression states have a relationship as same, one-away, or not one-away by
comparing all the binary outputs of microarray test of the first biological sample with the microarray test of the second biological sample,
outputting that the two tests are identical when all the binary outputs of microarray test of the first biological sample are the same as the microarray test of the second biological sample,
outputting that the two tests are not one genetic event away when all binary outputs of microarray test of the first biological sample have a plurality of differences from the binary outputs of microarray test of the second biological sample, and
outputting that the two tests are one genetic event away when all binary outputs of microarray test of the first biological sample have one difference from the binary outputs of microarray test of the second biological sample.

22. The method according to claim 1, wherein the conducting laboratory tests to determine partial or complete nucleotide sequence or expression state data comprises in silico DNA sequencing, and the processing device determines whether the first and second nucleotide sequence or expression states have a relationship as same, one-away, or not one-away by
inputting the first sequence and determining a plurality of transformed sequences, the plurality of transformed sequences being determined by transforming each character of the first sequence into a new character;
outputting the plurality of transformed sequences;
storing a relationship between the first sequence and each of the plurality of transformed sequences as being one-away in the database;
comparing the second sequence to the database storing the relationship between the first sequence and the each of the plurality of transformed sequences;
outputting identical when the second sequence is identical to the first sequence;
outputting one away when the second sequence is identical to one of the plurality of transformed sequences; and
outputting not one-away when the second sequence is not identical to any of stored relationships in the database.

23. An Infection Control Analysis Decision System comprising a processing device in communication with memory containing instructions for carrying out the method of claim 1 for a plurality of pathogens in a healthcare facility and instructions for applying Bayesian statistical techniques to calculate the likelihood that a patient will acquire an infection from a pathogen with a specific molecular fingerprint based upon patient risk factors and the spatial-temporal density of each pathogen and to output specific actions for preventing the transmission of the pathogens.
